(19) United States
(12) Patent Application Publication
(10) Pub. No.: US 2025/0277042 A1
(43) Pub. Date: Sep. 4, 2025

(54) TREATMENT OF PRURITIS EMPLOYING ANTI-IL13Ra1 ANTIBODY OR BINDING FRAGMENT THEREOF

(71) Applicant: Aslan Pharmaceuticals Pte Ltd, Singapore (SG)

(72) Inventors: Karen Veverka, Kansas City, KS (US); Ferda Cevikbas, Palo Alto, CA (US)

(21) Appl. No.: 18/695,464

(22) PCT Filed: Sep. 27, 2022
(86) PCT No.: PCT/SG2022/050693

§ 371 Date: Mar. 26, 2024

Related U.S. Application Data
(63) Continuation-in-part of application No. 17/929,824, filed on Sep. 6, 2022.
(63) Continuation-in-part of application No. 17/929,874, filed on Sep. 6, 2022.
(60) Provisional application No. 63/323,961, filed on Mar. 25, 2022.
(60) Provisional application No. 63/375,736, filed on Sep. 15, 2022.
(60) Provisional application No. 63/376,663, filed on Sep. 22, 2022.

(30) Foreign Application Priority Data
Sep. 27, 2021 (SG) 10202110688T
Sep. 27, 2021 (SG) 10202110690S
Sep. 27, 2021 (SG) 10202110730R
Oct. 29, 2021 (SG) 10202112085U
Feb. 22, 2022 (SG) 10202201722U
May 18, 2022 (SG) 10202205222Q
Aug. 26, 2022 (SG) 10202250837V
Sep. 6, 2022 (SG) 10202250923N

(51) Int. Cl. C07K 16/28; A61K 39/00; A61P 17/04

(52) U.S. Cl. CPC C07K 16/2866; A61P 17/04; A61K 2039/505; A61K 2039/545

(57) ABSTRACT

An anti-IL13Rα1 antibody (for example eblasakimab), an antigen binding fragment thereof, or a pharmaceutical formulation comprising the same for use in the treatment of pruritus (itchy skin) in a patient.

### REFERENCE TO AN ELECTRONIC SEQUENCE LISTING

The instant application contains a Sequence Listing submitted via EFS-Web, hereby incorporated by reference in its entirety, which was created on Mar. 14, 2024, is named “ST.APL28.NP_seqlist.xml” and is 83,250 bytes in size and corresponds to the Sequence Listing originally submitted in International Appl. No. PCT/SG2022/050693.

The present disclosure relates to use of an anti-IL-13Rα1 antibody or a binding fragment thereof and pharmaceutical formulations comprising the same to treat patients with pruritis.

### BACKGROUND

Pruritus is a condition where the skin becomes itchy. It results in a very uncomfortable and irritating sensation that compels the individual to scratch the skin. Constant scratching of the skin in order to relief the itch can damage the skin, which reduces its effectiveness as a protective barrier and may lead to infections. Furthermore, scratching an affected area tends to make that area itchier, which leads to even more scratching, thereby resulting in a vicious cycle which is hard to break. Prolonged scratching over time may also result in lichenification (thickened skin) and excoriation (scratch marks). In severe cases, pruritus can even lead to disturbed sleep, anxiety and depression.

Cytokines are known to act as pruritogens (itch inducing substances) in mice and humans. For some cytokines such as IL-31 and IL-4, their role in pruritus has been well documented. However, for other cytokines, such as IL-13, there is less evidence of a direct role in the pathophysiology of pruritus. Surprisingly, the present inventors have established that that inhibiting IL-13Rα1 is able to block the neural pathways that generate itch and thereby reduce itch irrespective of the underlying causes. This an important finding. What is more the present inventors believe that these pathways may be related to neuropathic pain and therefore in an independent aspect the present disclosure relates to the treatment or amelioration of neuropathic pain.

Although pruritus is not a life-threatening medical condition, it has a pronounced and debilitating effect on quality of life. Itch is not specific to one particular disease, but instead is common to a number diseases.

Furthermore, chronic itch is a hallmark and major symptom of atopic dermatitis and other type 2-driven inflammatory skin disorders. Itch signaling in atopic dermatitis (AD) has been recently postulated to be exacerbated by pro-inflammatory cytokines present in the skin, causing an immune response that disrupts the skin barrier and drives disease pathology.

One way to inhibit the activity of IL-13 is to interfere with the binding of IL-13 to its receptor IL-13R, for example by using an antibody specific to IL-13R, such as an antibody specific to IL-13Rα1. An effective antibody antagonist to IL-13Rα1 may also interfere with the binding of IL-13 and prevent heterodimerization of IL-4Rα and IL-13Rα1. Such an antibody could inhibit signaling of both IL-13 and IL-4 through the type II receptor while sparing IL-4 signalling through the type I receptor. Signalling through the type I receptor is essential in the induction phase of the immune response during which Th2 cells differentiate.

T cells do not express IL-13Rα1 so the type II receptor plays no role in Th2 differentiation. Hence, an IL-13Rα1 antibody should not affect the overall Th1/Th2 balance.

Signalling through the type II IL-4/IL-13 receptor is critical during the effector −A-stage of the immune response during established allergic inflammation.

Antibodies against IL-13Rα1 (both monoclonal and polyclonal) have been described in the art; see, eg, WO 97/15663, WO 03/80675; WO 03/46009; WO 06/072564; Gauchat et al, 1998 Eur. J. Immunol. 28:4286-4298; Gauchat et al, 2000 Eur. J. Immunol. 30:3157-3164; Clement et al, 1997 Cytokine 9(11): 959 (Meeting Abstract); Ogata et al, 1998 J. Biol. Chem. 273:9864-9871; Graber et al, 1998 Eur. J. Immunol. 28:4286-4298; C. Vermot-Desroches et al, 2000 Tissue Antigens 5 (Supp. 1):52-53 (Meeting Abstract); Poudrier et al, 2000 Eur. J. Immunol. 30:3157-3164; Akaiwa et al, 2001 Cytokine 13:75-84; Cancino-Diaz et al, 2002 J. Invest. Dermatol. 119:1114-1120; and Krause et al, 2006 Mol. Immunol. 43:1799-1807.

One particularly promising anti-IL-13Rα1 antibody is described in WO2008/060813 as antibody 10G5-6. 10G5-6 as an IgG4 with a hinge stabilising serine to proline mutation (S241P Kabat numbering). This antibody is now known as Eblasakimab (previously called ASLAN004, both names used interchangeably herein). Eblasakimab has been shown to bind to human IL-13Rα1 with a high affinity (for example Kd may be 500 pM). Eblasakimab was shown to effectively antagonise IL-13 function through inhibiting the binding of IL-13 to its receptor IL-13Rα1 and to inhibit IL-13 and IL-4 induced eotaxin release in NHDF cells, IL-13 and IL-4 induced STAT6 phosphorylation in NHDF cells and IL-13 stimulated release of TARC in blood or peripheral blood mononuclear cells.

Existing treatments for pruritus are typically topical medicines, such as antihistamines, corticosteroid creams, calcineurin inhibitors such as tacrolimus, and topical anesthetics, such as capsaicin. However, in some instances, moderate to severe forms of pruritus are not adequately controlled by topical medicines. In addition, it is not advisable for some patients to take the available topical medicines.

Accordingly, there is a need for alternative treatments in order to manage and treat pruritus.

### SUMMARY OF THE DISCLOSURE

The following paragraphs summarise the present disclosure:
1. An antibody or antigen binding fragment thereof, which is an inhibitor of signalling through IL-13Rα1 by binding the said receptor, for use in the treatment of pruritus (such as neural itch) in a patient by parenteral administration of a treatment cycle comprising a dose in the range 200 mg to 600 mg, (such as 400 to 600 mg), for example wherein the disease is modified by a percentage reduction in pruritus numerical rating score (P-NRS), for example a percentage reduction in P-NRS for average itching and/or P-NRS for worst itching, in the range −15 to −100% from the baseline.
1A. An antibody or antigen binding fragment thereof, which is an inhibitor of signalling through IL-13Rα1 by binding the said receptor, for use in the treatment to reduce pruritus numerical rating score (P-NRS), for example to reduce P-NRS for average itching (average P-NRS) and/or P-NRS for worst itching (peak P-NRS), in the range −15 to −100% from the baseline in a patient with pruritus by parenteral administration of a treatment cycle comprising a dose in the range 200 mg to 600 mg, (such as 400 to 600 mg).
2. An antibody or antigen binding fragment thereof for use according to paragraph 1 or 1A, wherein the reduction in P-NRS is for average itching (average P-NRS).
3. An antibody or antigen binding fragment thereof for use according to paragraph 1 or 1A, wherein the reduction in P-NRS is for worst itching (peak or worst P-NRS).
4. An antibody or antigen binding fragment thereof for use according to any one of the preceding paragraphs, wherein the patient is a highly allergic patient, for example where the baseline baseline IgE levels have been established and are at a level of at least 10,000 KU/L+/−2,000.
5. An antibody or antigen binding fragment thereof for use according to paragraph 4 wherein the baseline IgE levels are in the range 10,000+/−2,000 to 30,000+/−6,000 KU/L.
6. An antibody or antigen binding fragment thereof for use according to any one of paragraphs 4 to 5, wherein the treatment reduces the IgE levels by at least 15% from baseline.
7. An antibody or binding fragment thereof for use according to paragraph 6, wherein the treatment reduces the IgE levels by at least 20% from the baseline.
8. An antibody or binding fragment thereof for use according to paragraph 6 or 7, wherein the treatment reduces the IgE levels by at least 30% from baseline, for example reduces said levels 30 to 40%, such as 30, 31, 32, 33, 34, 35, 36, 37, 38, 39 or 40% from baseline.
9. An antibody or binding fragment according to any one of paragraphs 4 to 8, wherein the reduction is observed by about day 15.
10. An antibody or binding fragment thereof according to any one of paragraphs 4 to 8, wherein the reduction is observed by about day 29.
11. An antibody or binding fragment thereof according to any one of paragraphs 4 to 10, wherein the reduction is observed by about day 57.
12. An antibody or antigen binding fragment thereof for use according to any one of the preceding paragraphs, wherein the pruritus is mediated by one or more of the following: histamine, 5-HT (serotonin), acetylcholine, substance P (SP), leukotrienes, bradykinin, proteases (such as typsin, tryptase, cathepsin S, or kallikrein-related peptidases such as KLK4 or KLK14), IL-31, lysophosphatidic acid, autotaxin and/or toll-like receptor 7 (TLR7).
13. An antibody or antigen binding fragment thereof for use according to any one of the preceding paragraphs, wherein the pruritus is mediated by histamine.
14. An antibody or antigen binding fragment thereof for use according to any one of the preceding paragraphs, wherein the pruritus is mediated by IL-13, IL-4 or a combination of both.
15. An antibody or antigen binding fragment thereof for use according to any one of the preceding paragraphs, wherein the pruritus is mediated by both IL-13 and IL-4.
16. An antibody or antigen binding fragment thereof for use according to any one of the preceding paragraphs, wherein the patient has a skin condition, for example selected from the group comprising dermatitis; such as atopic dermatitis (AD), contact dermatitis, neurodermatitis or seborrheic dermatitis; eczema; hand-foot-and-mouth disease; hives (including urticaria associated with Lupus), psoriasis, an infection, such as a fungal or bacterial infection, for example impetigo or folliculitis; an allergic skin reaction; Ehlers-Danlos syndrome; asthma; and angioedema, such as hereditary angioedema (HAE).
17. An antibody or antigen binding fragment thereof for use according to any one of the preceding paragraphs, wherein the patient has dermatitis, for example atopic dermatitis (AD), contact dermatitis, neurodermatitis or seborrheic dermatitis.
18. An antibody or antigen binding fragment thereof for use according to any one of the preceding paragraphs, wherein the patient has atopic dermatitis (AD), for example moderate to severe atopic dermatitis, including lesional AD or a patient that received prior treatment with dupilumab.
19. An antibody or antigen binding fragment thereof for use according to any one of the preceding paragraphs, wherein the patient has Ehlers-Danlos syndrome (EDS), asthma or angioedema (such as HAE).
20. An antibody or antigen binding fragment thereof for use according to any one of the preceding paragraphs, wherein the patient has asthma.
21. An antibody or antigen binding fragment thereof for use according to any one of the preceding paragraphs, wherein the patient has angioedema.
22. An antibody or antigen binding fragment thereof for use according to any one of paragraphs 1 to 15, wherein the patient does not have atopic dermatitis or psoriasis.
23. An antibody or antigen binding fragment thereof for use according to any one of paragraphs 1 to 15, wherein the patient does not have atopic dermatitis.
24. An antibody or antigen binding fragment thereof for use according to any one of paragraphs 1 to 15, wherein the patient does not have psoriasis.
25. An antibody or antigen binding fragment thereof for use according to any one of the preceding paragraphs, wherein a reduction in P-NRS is present after about two weeks from administration of the first dose (such as day 15).
26. An antibody or antigen binding fragment thereof for use according to any one of paragraphs 1 to 25, wherein a reduction in P-NRS is present after about four weeks from administration of the first dose (such as day 29).
27. An antibody or antigen binding fragment thereof for use according to any one of paragraphs 1 to 26, wherein a reduction in P-NRS is present after about six weeks from administration of the first dose (such as day 43).
28. An antibody or antigen binding fragment thereof for use according to any one of paragraphs 1 to 27, wherein a reduction in P-NRS is present after about eight weeks from administration of the first dose (such as day 57).
29. An antibody or antigen binding fragment thereof for use according to any one of the preceding paragraphs, wherein the treatment is administered intravenously.
30. An antibody or antigen binding fragment thereof for use according to any one of the preceding paragraphs, wherein the treatment is administered subcutaneously.
31. An antibody or antigen binding fragment thereof for use according to any one of the preceding paragraphs, wherein multiple doses are administered in a treatment cycle (for example wherein the treatment cycle is 4 to 8 weeks, such as 8 weeks).
32. An antibody or antigen binding fragment thereof for use according to any one of the preceding paragraphs, wherein multiple treatment cycles are administered, for example 2, 3, 4 or more treatment cycles are administered.
33. An antibody or antigen binding fragment thereof for use according to paragraphs 31 or 32 wherein following the treatment cycle or cycles and disease modification, maintenance therapy is administered, for example the same dose administered less frequently (for example monthly), or a lower dose (such as 200 mg) administered the same frequency or less frequently (such as about two weekly, about three weekly, or about four weekly.
34. An antibody or antigen binding fragment thereof for use according to any one of the preceding paragraphs, wherein said antibody or binding fragment thereof is administered approximately weekly, (in particular a single treatment cycle, especially 8 weeks).
35. An antibody or antigen binding fragment thereof for use according to any one of paragraphs 1 to 33, wherein said antibody or binding fragment thereof is administered once approximately every two weeks, (in particular in a treatment cycle (including a single cycle), especially a cycle of 8 weeks).
36. An antibody or antigen binding fragment thereof for use according to any one of paragraphs 1 to 33, wherein said antibody or binding fragment thereof is administered once approximately every three weeks, (in particular in a treatment cycle (including a single cycle), especially a cycle of 8 weeks).
37. An antibody or antigen binding fragment thereof for use according to any one of paragraphs 1 to 33, wherein the antibody or binding fragment thereof is administered once approximately every four weeks (for example monthly), (in particular in a treatment cycle (including a single cycle), especially a cycle of 8 weeks).
38. An antibody or antigen binding fragment thereof for use according to any one of the preceding paragraphs, wherein a loading dose, for example in the range 400 to 900 mg, such as 400, 500, 600, 700, 800 or 900 mg, is employed before administration of the treatment cycle.
39. An antibody or antigen binding fragment thereof for use according to any one of paragraphs 1 to 37, wherein the treatment does not comprise a loading dose.
40. An antibody or antigen binding fragment thereof for use according to any one of the preceding paragraphs, wherein the dose is 200 mg.
41. An antibody or antigen binding fragment thereof for use according to any one of paragraphs 1 to 39, wherein the dose is 400 mg.
42. An antibody or antigen binding fragment thereof for use according to any one of paragraphs 1 to 39, wherein the dose is 600 mg.
43. An antibody or antigen binding fragment thereof for use according any one of the preceding paragraphs, wherein the reduction in P-NRS score is at least −20%, such as at least −25%, at least −30%, at least −35% or at least −40%.
44. An antibody or antigen binding fragment thereof for use according to any one of the preceding paragraphs, wherein the reduction in P-NRS score is at least −35%.
45. An antibody or antigen binding fragment thereof for use according to any one of the preceding paragraphs, wherein the reduction in P-NRS score is in the range −20 to −70%, for example −20 to −29%, −30 to −39%, −40 to −49% −50 to−59%, or −60 to −69%.
46. An antibody or antigen binding fragment thereof for use according to any one of paragraphs 1 to 42, wherein the reduction in P-NRS is in the range −15 to −25% (for example at about day 15).
47. An antibody or antigen binding fragment thereof for use according to any one of paragraphs 1 to 42, wherein the reduction in P-NRS is in the range −25 to −35% (for example at about day 29).
48. An antibody or antigen binding fragment thereof for use according to any one of paragraphs 1 to 42, wherein the reduction in P-NRS is in the range −35 to −45% (for example at about day 43 or 57).
49. An antibody or antigen binding fragment thereof for use according to any one of the preceding paragraphs, wherein the treatment cycles comprises, a first dose at 600 mg, followed by three weekly doses of 400 mg, for example wherein the treatment cycle is repeated twice i.e. two treatment cycles lasting 8 weeks, in particular day 1 600 mg, approximately day 8 400 mg, approximately day 15 400 mg, approximately day 22 400 mg, approximately day 29 600 mg, approximately day 36 400 mg, approximately day 43 400 mg, and approximately day 50 400 mg are administered.
50. An antibody or antigen binding fragment thereof for use according to any one of the preceding paragraphs, wherein disease modification, occurs by day 8, wherein day 1 is the first administration of the antibody or binding fragment thereof.
51. An antibody or antigen binding fragment thereof for use according to any one of the preceding paragraphs, wherein the disease modification is a reduction in peak P-NRS, for example wherein the reduction is a percentage from base line in the range −15 to −70%.
52. An antibody or antigen binding fragment thereof for use according to any one of the preceding paragraphs, wherein the disease modification in the range −30 to −100% is achieved by about day 57 following first administration on day 1, for example maximum disease modification is achieved by about day 57.
53. An antibody or antigen binding fragment thereof for use according to any one of the preceding paragraphs, wherein the antibody or binding fragment binds an epitope FFYQ (for example same epitope as the antibody with a VH shown in SEQ ID NO: 51 and a VL shown in SEQ ID NO: 53, or a sequence at least 95% identical to any one of the same.
54. An antibody or antigen binding fragment thereof for use according to any one of the preceding paragraphs, wherein the anti-IL-13R antibody comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: 10.
55. An antibody or antigen binding fragment thereof for use according to any one of the preceding paragraphs, wherein the anti-IL-13R antibody comprises a VH domain comprising an amino acid sequence shown in SEQ ID NO: 51 or a sequence at least 95% identical thereto, in particular SEQ ID NO: 51.
56. An antibody or antigen binding fragment thereof for use according to any one of the preceding paragraphs, wherein the anti-IL-13R antibody comprises a VL CDR1 comprising an amino acid sequence as set forth in SEQ ID NO: 31, a VL CDR2 comprising an amino acid sequence as set forth in SEQ ID NO: 32, and a VL CDR3 comprising an amino acid sequence as set forth in SEQ ID NO: 45.
57. An antibody or antigen binding fragment thereof for use according to any one of the preceding paragraphs, wherein the anti-IL-13R antibody comprises a VL domain comprising an amino acid sequence shown in SEQ ID NO: 53 or a sequence at least 95% identical thereto, in particular SEQ ID NO: 53.
58. An antibody or antigen binding fragment thereof for use according to any one of the preceding paragraphs, wherein the anti-IL-13R antibody comprises a VL domain comprising an amino acid sequence shown in SEQ ID NO: 53 or a sequence at least 95% identical thereto, in particular SEQ ID NO: 53, and a VH comprising an amino acid sequence shown in SEQ ID NO: 51 or a sequence at least 95% identical thereto, in particular SEQ ID NO: 51.
59. A pharmaceutical formulation comprising an antibody or binding fragment for use according to any one of paragraphs 1 to 58, said formulation comprising: 10 to 140 mg/ml of the antibody or binding fragment (for example 10, 15, 20, 25, 30, 35, 40, 45, 50, 55, 60, 65, 70, 75, 80, 85, 90, 95, 100, 105, 110, 115, 120, 125, 130, 135, 140 mg/ml); 50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine); 15 to 25 mM histidine buffer, for example 15, 16, 17, 18, 19, 20, 21, 22, 23, 24 and 25, such as 20 mM histidine buffer; 0.01-0.03% of a non-ionic surfactant, such as 0.02% w/v and the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9).
60. A pharmaceutical formulation for use according to paragraph 59 wherein said formulation comprises: 10 to 140 mg/ml of the antibody or binding fragment; 50 mM to 150 mM of arginine (for example 50, 55, 60, 65, 70, 75, 80, 85, 90, 95, 100, 105, 110, 115, 120, 125, 130, 135, 140, 145 or 150, such as 100 mM arginine); 15 to 25 mM histidine buffer, for example 15, 16, 17, 18, 19, 20, 21, 22, 23, 24 and 25, such as 20 mM histidine buffer; 0.01-0.03% of a non-ionic surfactant, such as 0.02% w/v and wherein the pH of the formulation is in the range 5.5 to 7.5 for example 6.2 to 7.2 (such as 6.2, 6.3, 6.4, 6.5, 6.6, 6.7, 6.8, 6.9, 7.0, 7.1, 7.2), such as 6.5 to 7.0, in particular 6.4 to 6.9)
61. A pharmaceutical formulation comprising the antibody or binding fragment thereof according to any one of paragraphs 1 to 58, said formulation comprising 150 to 210 mg/ml of an anti-IL-13R antibody or antigen binding fragment thereof, for example 150, 155, 160, 165, 170, 175, 180, 185, 190, 195, 200, 205 or 210 mg/ml, in particular 150 mg/ml, 175 mg/ml or 200 mg/ml;
   170 to 250 mM of arginine (such as Arg-HCl or Arg-Glu), for example 170, 175, 180, 185, 190, 195, 200, 205, 210, 215, 220, 225, 230, 235, 240, 245 or 250 mM, in particular 150 mM, 175 mM or 250 mM;
   20 to 50 mM histidine buffer, for example 20, 25, 30, 35, 40, 45 or 50 mM, such as 20 mM or 50 mM histidine buffer;
   0.01-0.03% of a non-ionic surfactant, such as 0.02% w/w; and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.5.
62. A pharmaceutical formulation comprising an antibody or binding fragment thereof according to any one of paragraphs 1 to 58, said formulation comprising 175 to 250 mg/ml of an anti-IL-13R antibody or antigen binding fragment thereof, for example 175, 180, 185, 190, 195, 200, 205, 210, 215, 220 or 225 mg/ml, in particular 200 mg/ml, 225 mg/ml or 250 mg/ml;
   15 to 75 mM of tryptophan, such as 15 to 60 mM, in particular 25 to 50 mM tryptophan;
   190 to 270 mM of arginine (such as Arg-HCl or Arg-Glu), for example 190, 195, 200, 205, 210, 215, 220, 225, 230, 235, 240, 245, 250, 255, 260, 265 or 270 mM, in particular 200, 210, 225, 235, 250 or 260 mM;
   0.01-0.03% of a non-ionic surfactant, for example 0.01-0.03% w/w, such as 0.02% w/w; a buffer (such as histidine buffer); and
   wherein the pH of the formulation is in the range 6.0 to 7.0, such as 6.0, 6.1, 6.2, 6.3, 6.4, 6.5, 6.6, 6.7, 6.8, 6.9 or 7.0, in particular 6.4.
63. A pharmaceutical formulation for use according to any one of paragraphs 59 to 62, wherein the osmolarity of the formulation is in the range 350 to 550 mOsmo/kg, for example 350, 355, 365, 370, 375, 380, 385, 390, 395, 400, 405, 410, 415, 420, 425, 430, 435, 440, 445, 450, 455, 460, 465, 470, 475, 480, 485, 490, 495, 500, 505, 515, 520, 525, 530, 535, 540, 545, 550, such as 405 to 435 mOsmo/kg.
64. A pharmaceutical formulation for use according to any one of paragraphs 59 to 63, which further comprises 50 to 200 mM of a sugar, for example 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, such as 180 mM sugar.
65. A pharmaceutical formulation for use according to any one of paragraphs 59 to 64, wherein the pH is 6.5.
66. A pharmaceutical formulation for use according to any one of paragraphs 59 to 65, wherein the formulation does not comprise NaCl.
67. A pharmaceutical formulation for use according to any one of paragraphs 59 to 65, wherein the formulation comprises 50 to 150 mM of NaCl, for example50, 55, 60, 65, 70, 75, 80, 85, 90, 95, 100, 105, 110, 115, 120, 125, 130, 135, 140, 145, 150, such as 62.5 or 140 mM NaCl.
68. A method of treating a patient having pruritus (comprising administering parenterally an antibody, antigen binding fragment thereof or a pharmaceutical formulation, which is an inhibitor of signalling through of the IL-13Rα1 by binding the said receptor, for example according to any one of paragraphs 1 to 67, by parenteral administration of a treatment cycle comprising a dose in the range 200 mg to 600 mg, (such as 400 to 600 mg), wherein the disease is modified by a percentage reduction in pruritus numerical rating score (P-NRS), for example a percentage reduction in P-NRS for average itching and/or P-NRS for worst itching, in the range −15 to −100% from the baseline.
69. Use of an antibody, antigen binding fragment or a pharmaceutical formulation, which is an inhibitor of signalling through of the IL-13Rα1 by binding the said receptor, for example according to any one of paragraphs 1 to 67, for use in the manufacture of a medicament for the treatment of pruritus in a patient by parenteral administration of a treatment cycle comprising a dose in the range 200 mg to 600 mg, (such as 400 to 600 mg), wherein the disease is modified by a percentage reduction in pruritus numerical rating score (P-NRS), for example a percentage reduction in P-NRS for average itching and/or P-NRS for worst itching, in the range −15 to −100% from the baseline.
70. An antibody or antigen binding fragment thereof, which is an inhibitor of signalling through IL-13Rα1 by binding the said receptor, for use in the treatment of neuropathic pain or neuropathy, such as peripheral neuropathic pain, central neuropathic pain, or mixed (peripheral and central) neuropathic pain.
71. Use of an antibody or antigen binding fragment thereof, which is an inhibitor of signalling through IL-13Rα1 by binding the said receptor, in the manufacture of a medicament for the treatment of neuropathic pain or neuropathy, such as peripheral neuropathic pain, central neuropathic pain, or mixed (peripheral and central) neuropathic pain.
72. A method of treating a patient having neuropathic pain or neuropathy, such as peripheral neuropathic pain, central neuropathic pain, or mixed (peripheral and central) neuropathic pain, comprising administration of an antibody or antigen binding fragment, which is an inhibitor of signalling through IL-13Rα1 by binding the said receptor.
73. An antibody or antigen binding fragment thereof for use, a use or a method according to any one of paragraphs 70 to 72, wherein the neuropathic pain is caused by a neuropathy, for example wherein the neuropathy is caused by one of the following: diabetes; trauma; autoimmune disorders such as Gullain-Barre syndrome, rheumatoid arthritis, Sjoren's syndrome; infections such as chickenpox, shingles, HIV, herpes, Epstein-Barr virus, West Nile virus and hepatitis C; kidney disorders; liver disorders; hypothyroidism; tumors (malignant or benign) that compress nerves; medications or poisons such as chemotherapy and radiation; vascular disorders; alcoholism; and inherited disorders such as Charcot-Marie Tooth disease, Fabry disease, and familial amyloidosis.
74. An antibody or antigen binding fragment thereof for use, a use or a method according to any one of paragraphs 70 to 73, wherein the neuropathic pain is selected from the group comprising: peripheral neuropathic pain, for example peripheral neuropathic pain in the feet, legs, arms or hands; autonomic neuropathic pain; focal neuropathic pain, for example focal neuropathic pain in the head, hand, torso or leg, Bell's palsy; proximal neuropathic pain; neuropathic pain associated with diabetes; neuropathic pain associated with compression mononeuropathic neuropathy, such as Carpal tunnel syndrome; Phantom limb syndrome; trigeminal neuralgia; neuropathic pain associated with thoracic or lumbar radiculopathy; neuropathic pain associated with an infection, such as shingles, HIV or syphilis; sciatica; neuropathic pain associated with amputation; neuropathic pain associated with spine surgery; and neuropathic pain associated with multiple myeloma
75. An antibody or antigen binding fragment thereof for use, a use or a method according to any one of paragraphs 70 to 74, comprising a dose in the range 200 mg to 600 mg of the antibody or antigen binding fragment thereof.
76. An antibody or antigen binding fragment thereof for use, a use or a method according to any one of paragraphs 70 to 75, wherein the antibody or antigen-binding fragment thereof is as defined in any one of paragraphs 53 to 58.
77. An antibody or antigen binding fragment thereof for use, a use or a method according to any one of paragraphs 70 to 76, wherein the treatment is as defined in any one of paragraphs 29 to 42.

Surprisingly, the present inventors have established that the symptoms and severity of pruritus can be treated and/or ameliorated by administering eblasakimab to subjects in need thereof.

In one embodiment there is provided a method of treating a patient having pruritus, such as a highly allergic patient as defined herein with an antibody or binding fragment thereof or a pharmaceutical formulation as defined herein.

In one embodiment the itch is caused by an infection, such as viral infection, bacterial infection, fungal infection or a parasitic infection. In one embodiment the viral infection is chickenpox.

In one embodiment there is provided use of an antibody or binding fragment thereof or a pharmaceutical formulation, as defined herein for the manufacture of a medicament for the treatment of pruritus in a patient, for example in a highly allergic patient.

In one embodiment, the treatment with eblasakimab results in a significant reduction in pruritis score, for example a reduction in pruritus numerical score (P-NRS) in the range of −15 to −100% from the baseline.

In one embodiment the patient has a type 2-driven inflammatory skin disorder, for example a skin disorder exacerbated by pro-inflammatory cytokines present in the skin. Thus, in one embodiment eblasakimab is suitable for treating a patient having a type-2 driven inflammatory skin disorder. In one embodiment the type 2-driven inflammatory skin disorder is atopic dermatitis (AD).

In one embodiment itch signalling in the patient is exacerbated by pro-inflammatory cytokines present in the skin of the patient, which may cause an immune response that disrupts the skin barrier and drives disease pathology. Thus, in one embodiment eblasakimab is able to reduce or inhibit itch signalling in a patient. In one embodiment treatment with eblasakimab prevents or reduces the disruption of the skin barrier in a patient. In one embodiment eblasakimab dampens or inhibits the immune response that drives disease pathology in a patient having pruritus.

In one embodiment the pro-inflammatory cytokines are IL-13, IL-4 or a combination of both IL-13 and IL4. In one embodiment IL-13, IL-4 or both act as neuronal enhancers for the amplification of itch pathways through the 13Rα1 subunit of the Type-2 receptor. Thus, in one embodiment eblasakimab inhibits or dampens neuronal enhancers for the amplification of itch pathways, such as IL-13 and/or IL-4.

In one embodiment treatment with eblasakimab reduces the overall burden of disease, for example reduces the overall burden of atopic dermatitis in a patient.

In one embodiment treatment with eblasakimab improves quality of life in a patient having itch, for example a highly allergic patient.

In one embodiment treatment with eblasakimab results in a reduction of pruritic neuronal response, for example via eblaskimab's dual blockade of both IL-4 and IL-13 through the Type 2 receptor.

In one embodiment a combination therapy is employed comprising the antibody, antigen binding fragment thereof or a formulation according to the present disclosure and a further medicament. In one embodiment the further medicament is for the treatment of pruritus, for example topical steroids, oral steroids, and/or antihistamines.

Surprisingly disease modification following treatment with an anti-IL-13Rα1 antibody or binding fragment thereof according to the present disclosure closely follows reduction in TARC, in fact the TARC reduction and EASI reduction correlate closely.

In one embodiment the patient has a skin condition, for example selected from the group comprising dermatitis; such as atopic dermatitis (AD), contact dermatitis, neurodermatitis or seborrheic dermatitis; eczema; hand-foot-and-mouth disease; hives (including urticaria associated with Lupus), psoriasis, an infection, such as a fungal or bacterial infection, for example impetigo or folliculitis; an allergic skin reaction; Ehlers-Danlos syndrome; asthma; and angioedema, such as hereditary angioedema (HAE).

In one embodiment the patient has dermatitis, for example atopic dermatitis (AD), contact dermatitis, neurodermatitis or seborrheic dermatitis.

In one embodiment the patient has atopic dermatitis (AD), for example moderate to severe atopic dermatitis.

In one embodiment the patient has eczema.

In one embodiment the patient has hives.

In one embodiment the patient has Ehlers-Danlos syndrome (EDS), asthma or angioedema (such as HAE).

In one embodiment the patient has Ehlers-Danlos syndrome.

In one embodiment the patient has asthma.

In one embodiment the patient has angioedema, such as HAE.

The present inventors have also established that the antibodies, antigen binding fragments and compositions of present invention can be employed to treated other diseases with an allergic component, for example allergic epithelial disease, such as allergic asthma, asthma-COPD and eosinophilic esophagitis.

Thus, in an independent aspect there is provided treatment of an allergic disease, for example with elevated IgE levels (elevated in comparison to normal levels), with an antibody, binding fragment or composition comprising the same, which is an inhibitor of signalling through IL-13Rα1, by binding the said receptor, for example as described elsewhere herein (for atopic dermatitis).

Whilst not wishing to be bound by theory, the present inventors believe that the same cytokines and similar or corresponding mechanisms are at work in these allergic diseases as in this population of atopic dermatitis.

In one embodiment the allergic disease is manifested in epithelial tissue.

In one embodiment the allergic disease is allergic asthma, for example poorly controlled and/or moderate to severe asthma.

In one embodiment the allergic disease is asthma-COPD, for example poorly controlled and/or moderate to severe asthma-COPD.

In one embodiment the allergic disease is eosinophilic esophagitis, for example poorly controlled and/or moderate to severe eosinophilic esophagitis.

In one embodiment the patient is identified as allergic before treatment, for example where the baseline has been established and is at a level of at least 10,000 KU/L+/−2,000.

Preferences described herein for atopic dermatitis, like dose and the like apply equally to allergic disease indications.

In one embodiment the allergic disease is not atopic dermatitis.

In one embodiment the allergic disease is not eosinophilic esophagitis.

In one embodiment the allergic disease is not psoriasis.

Whilst not wishing to be bound by theory, the present inventors have demonstrated that IL13 and IL-4 act as neuronal enhancers for a wide range of itch pathways, and that eblasakimab was able to reduce neuronal spontaneous activity. Thus, by inhibiting Il-13 Rα1 signaling by using an anti-IL13Rα1 antibody or antigen binding fragment, such as eblasakimab, it may be possible to modulate the neuronal activity of other pathways, in particular neuropathic pain pathways.

Thus, in an independent aspect, there is provided an antibody or antigen binding fragment thereof, which is an inhibitor of signalling through IL-13Rα1 by binding the said receptor, for use in the treatment of neuropathic pain, such as peripheral neuropathic pain, central neuropathic pain, or mixed (peripheral and central) neuropathic pain, for example comprising an anti-IL13Rα1 antibody or antigen-binding fragment as defined below.

### DETAILED DISCLOSURE

Disease modification as employed herein relates to improvements in the disease status for example as measured by a clinically relevant score, in particular a reduction in the peak pruritus numerical score (P-NRS).

Pruritus or pruritis as used herein refers to a condition where the skin becomes itchy. It results in a very uncomfortable and irritating sensation that compels the individual to scratch the skin. Pruritus can be caused by a range of different conditions, including skin conditions (such as atopic dermatitis and psoriasis), internal diseases (such as liver and kidney disease, lymphoma), nerve disorders (such as multiple sclerosis), psychiatric conditions (such as anxiety and obsessive compulsive disorder), and allergic reactions.

Pruritus numerical score/scale (P-NRS) as used herein is a patient assessed score of the intensity of his or her itch over the previous 24 hours based on a scale of 0 to 10, with a score of 0 referring to “no itch” and a top score of 10 referring to “worst imaginable itch”.

Average pruritus numerical score/scale (average P-NRS) as used herein refers a patient assessed score of the average intensity of his or her itch over the previous 24 hours based on a scale of 0 to 10, with a score of 0 referring to “no itch” and a top score of 10 referring to “worst imaginable itch”.

Peak pruritus numerical score/scale (peak P-NRS) or worst pruritus numerical score/scale (worst P-NRS) as used herein refers a patient assessed score of the worst intensity of his or her itch over the previous 24 hours based on a scale of 0 to 10, with a score of 0 referring to “no itch” and a top score of 10 referring to “worst imaginable itch”.

In one embodiment the reduction in P-NRS is a reduction in average P-NRS.

In one embodiment the reduction in P-NRS is a reduction in peak P-NRS or worst P-NRS.

In one embodiment the reduction in P-NRS is a reduction in average and peak P-NRS or worst P-NRS.

In one embodiment the reduction in average and/or peak P-NRS is in the range of −15% to −100%, for example −15%, −20%, −25%, −30%, −35%, −40%, −45%, −50%, −55%, −60%, −65%, −70%, −75%, −80%, −85%, −90%, −95% or −100%.

In one embodiment the reduction in average and/or peak P-NRS is in the range of −20% to −100%, for example −20%, −25%, −30%, −35%, −40%, −45%, −50%, −55%, −60%, −65%, −70%, −75%, −80%, −85%, −90%, −95% or −100%.

In one embodiment the reduction in average and/or peak P-NRS is in the range of −25% to −100%, for example −25%, −30%, −35%, −40%, −45%, −50%, −55%, −60%, −65%, −70%, −75%, −80%, −85%, −90%, −95% or −100%.

In one embodiment the reduction in average and/or peak P-NRS is in the range of −35% to −100%, for example −30%, −35%, −40%, −45%, −50%, −55%, −60%, −65%, −70%, −75%, −80%, −85%, −90%, −95% or −100%.

In one embodiment the reduction in average and/or peak P-NRS is in the range of −40% to −100%, for example −40%, −45%, −50%, −55%, −60%, −65%, −70%, −75%, −80%, −85%, −90%, −95% or −100%.

In one embodiment the reduction in average and/or peak P-NRS is in the range of −45% to −100%, for example −45%, −50%, −55%, −60%, −65%, −70%, −75%, −80%, −85%, −90%, −95% or −100%.

In one embodiment the reduction in average and/or peak P-NRS is in the range of −50% to −100%, for example −50%, −55%, −60%, −65%, −70%, −75%, −80%, −85%, −90%, −95% or −100%.

In one embodiment the reduction in average and/or peak P-NRS is in the range of −55% to −100%, for example −55%, −60%, −65%, −70%, −75%, −80%, −85%, −90%, −95% or −100%.

In one embodiment the reduction in average and/or peak P-NRS is in the range of −60% to −100%, for example −60%, −65%, −70%, −75%, −80%, −85%, −90%, −95% or −100%.

In one embodiment the reduction in average and/or peak P-NRS is in the range of −65% to −100%, for example −65%, −70%, −75%, −80%, −85%, −90%, −95% or −100%.

In one embodiment the reduction in average and/or peak P-NRS is in the range of −70% to −100%, for example −70%, −75%, −80%, −85%, −90%, −95% or −100%.

In one embodiment the reduction in average and/or peak P-NRS is in the range of −75% to −100%, for example −75%, −80%, −85%, −90%, −95% or −100%.

In one embodiment the reduction in average and/or peak P-NRS is in the range of −80% to −100%, for example −80%, −85%, −90%, −95% or −100%.

In one embodiment the reduction in average and/or peak P-NRS is in the range of −85% to −100%, for example −85%, −90%, −95% or −100%.

In one embodiment the reduction in average and/or peak P-NRS is in the range of −90% to −100%, for example −90%, −95% or −100%.

In one embodiment the reduction in average and/or peak P-NRS is in the range of −95% to −100%, for example −95% or −100%.

In one embodiment the reduction in average and/or peak P-NRS is in the range of −15% to −25%, for example −15%, −16%, −17%, −18%, −19%, −20%, −21%, −22%, −23%, −24% or −25%.

In one embodiment the reduction in average and/or peak P-NRS is in the range of −25% to −35%, for example −25%, −26%, −27%, −28%, −29%, −30%, −31%, −32%, −33%, −34% or −35%.

In one embodiment the reduction in average and/or peak P-NRS is in the range of −35% to −45%, for example −35%, −36%, −37%, −38%, −39%, −40%, −41%, −42%, −43%, −44% or −45%.

In one embodiment the reduction in average and/or peak P-NRS is in the range of 45% to −55%, for example −45%, −46%, −47%, −48%, −49%, −50%, −51%, −52%, −53%, −54% or −55%.

In one embodiment the reduction in average and/or peak P-NRS is in the range of 55% to −65%, for example −55%, −56%, −57%, −58%, −59%, −60%, −61%, −62%, −63%, −64% or −65%.

In one embodiment the reduction in average and/or peak P-NRS is in the range of 65% to −75%, for example −65%, −66%, −67%, −68%, −69%, −70%, −71%, −72%, −73%, −74% or −75%.

In one embodiment the reduction in average and/or peak P-NRS is in the range of 75% to −85%, for example −75%, −76%, −77%, −78%, −79%, −80%, −81%, −82%, −83%, −84% or −85%.

In one embodiment the reduction in average and/or peak P-NRS is in the range of 85% to −95%, for example −85%, −86%, −87%, −88%, −89%, −90%, −91%, −92%, −93%, −94% or −95%.

In one embodiment the reduction in average and/or peak P-NRS is in the range of 95% to −100%, for example −95%, −96%, −97%, −98%, −99%, or −100%.

A clinically relevant score is a score used in the clinic, for example used by a physician.

In one embodiment the disease is modified by a percentage reduction in Eczema Area and Severity Index (EASI) score in the range −20 to −100% from the baseline, such as EASI 50, EASI 75 or EASI 90. EASI score and EASI are used interchangeably herein.

Eczema Area and Severity Index (EASI) score as used herein is a tool used to measure the area (which indicates the extent of disease) and severity of atopic eczema. The number after the term “EASI” indicates the % decrease in the score from baseline. Thus, EASI 50 for example refers to 50% decrease in the score and EASI 90 refers to a 90% decrease in the score.

In one embodiment disease modification is measured as a reduction in IGA.

In one embodiment there is a provided a reduction in the Investigator Global Assessment (IGA) with/after treatment according to the present disclosure, for example an assessment of 0, 1 or 2, (no inflammatory signs, almost clear and mild disease respectively). In particular there is provided an IGA score of 0 or 1.

Investigator's global assessment (IGA) as used herein refers to a tool for the assessment of atopic dermatitis. It uses a 0-5 point scale depending on the severity of a patient's symptoms:

| Score | Severity | Description of symptoms |
|---|---|---|
| 0 | clear | no inflammatory signs |
| 1 | almost clear | Just perceptible erythema and just |
| | | perceptible papulation/infiltration |
| 2 | mild disease | Mild erythema and mild papulation/infiltration |
| 3 | moderate disease | moderate erythema and moderate papulation/infiltration |
| 4 | severe disease | severe erythema and severe papulation/infiltration |
| 5 | very severe disease | severe erythema and severe papulation/ |
| | | infiltration with oozing/crusting |

In an independent aspect there is provided use of an antibody, antigen binding fragment or pharmaceutical formulation as disclosed herein for the treatment or amelioration of allergic food responses, in particular severe allergic food response, such as peanut allergy.

In one embodiment, the highly allergic patient's baseline IgE levels have been established and are at a level of at least at least 10,000 KU/L+/−2,000, such as 8000, 8500, 9000, 9500, 10000, 10500, 11000, 11500 or 12000 KU/L.

In one embodiment, the patient has been identified as a highly allergic patient before the treatment is administered, for example, wherein the patient's baseline IgE levels have been established and are at a level of at least 10,000 KU/L+/−2,000. Thus, in one embodiment the patient is identified as a highly allergic patient wherein the patient's baseline IgE levels have been established and are at a level of at least 10,000 KU/L+/−2,000, prior to administration of the antibody or binding fragment thereof, pharmaceutical formulation or medicament as defined herein. Accordingly, in one embodiment the target population to be treated is highly allergic patients whose baseline IgE levels have been established and are at a level of at least 10,000 KU/L+/−2,000.

In one embodiment the patient is identified as highly allergic by clinical observation.

Asthma as used herein is a respiratory disease characterized by inflammation and bronchospasm, wherein the muscles around the airways tighten and contract in an attempt to keep the airways open. This leaves patients with cough, wheezing, chest tightness and shortness of breath. When the breathing issues become severe, this is typically referred to as an asthma attack.

Allergic asthma (used interchangeably with allergy-induced asthma) as used herein refers to form of asthma whereby the lungs of a patient become inflamed, and the airways tighten in response to the inhalation of an allergen. Common allergens include pollen, dust, animal dander and mold. Patients with allergic asthma experience many of the same symptoms as patients with non-allergic asthma—cough, wheezing, chest tightness and shortness of breath. Hence, the major difference between the two conditions is that patients with allergic asthma normally experience symptoms after inhaling an allergen.

Chronic obstructive pulmonary disease (COPD) as used herein is a collection of chronic inflammatory lung diseases that obstruct airflow and result in breathing problems. Emphysema and chronic bronchitis are the two most common conditions that contribute to COPD. Empysema is a condition wherein the alveoli are damaged and chronic bronchitis is a condition whereby the bronchial tubes are inflamed. These two conditions usually occur together and can vary in severity among individuals with COPD.

Asthma-COPD (used interchangeably with asthma-COPD overlap syndrome (ACOS)) as used herein refers to a condition whereby a patient has both asthma and COPD. Patients diagnosed with asthma-COPD have reduced lung function, have more severe asthma attacks and typically experience symptoms more frequently than patients with asthma or COPD alone. Hence, asthma-COPD is a more serious and dangerous condition than either disease alone.

Eosinophilic esophagitis (EOE) as used herein refers to a chronic immune system disease where eosinophils build up in the esophagus (eosinophils are not normally found in the esophagus). This build-up occurs as a reaction to foods, allergens or acid reflux, and may inflame and cause injury to the esophageal tissue. This in turn may lead to narrowing of the eosphagus and difficulty swallowing. In serious cases it may even result in medical emergencies due to food getting stuck in the throat. The majority of patients with EOE are atopic. Thus, individuals with atopic dermatitis, food or other environmental allergies are at greater risk of developing EOE. Family history of EOE is also a risk factor for the condition. No medications are currently approved by the US FDA for treating EOE. However, proton pump inhibitors (PPIs) have been demonstrated to reduce esophageal inflammation in some EOE patients and are thus often used as a first treatment. Corticosteroids may also be administered to help control inflammation.

Atopic dermatitis (AD) as used herein refers to inflammation of the skin and includes: dry/itchy skin and red rashes. AD can be a very painful, demoralising and psychologically damaging disease. The most common side effect of AD is pruritus, and a diagnosis of active AD cannot be made without an accompanying history of itching. Indeed, AD patients often complain that the pruritus is the most annoying and hardest symptom they have to cope with.

Psoriasis as used herein refers to a skin disorder that causes skin cells to multiply much faster than normal. This results in red, itchy scaly patches, which are typically found on the scalp, knees, elbows, and trunk. There are different categories of psoriasis, including pustular psoriasis, guttate psoriasis, inverse psoriasis and erythrodermic psoriasis. Most types of psoriasis go through cycles, flaring for a few weeks or months, then subsiding for a time or even going into remission.

Angioedema as used herein refers to the rapid edema (swelling) of the area beneath the skin or mucosa caused by the build-up of fluid, typically in response to an allergic trigger. Angioedema can affect different parts of the body, but it typically manifests in the eyes, lips, genitalia, hands and feet. Angioedema can arise with hives or alone. In more serious cases, angioedema can result in breathing difficulties, abdominal pain and dizziness.

Ehlers-Danlos syndrome (EDS) as used herein refers to a group of inherited disorders wherein the connective tissues, such as tendons, ligaments, blood vessels, etc are affected. There are 13 types of EDS, including hypermobile EDS (the most common), classical EDS, vascular EDS and kyphoscoliotic EDS. The different types of EDS share some common symptoms such as increased range of joint mobility (hypermobility), and stretchy and fragile skin. There are no known cures for EDS and treatment is largely supportive in nature, for example physiotherapy.

Hives (urticaria) as used herein refers to a sudden outbreak of itchy pale red bumps or welts on the skin. There are several types of hives including acute urticaria which last less than 6 weeks and are commonly caused by foods, medication or infections; chronic urticaria which last more than 6 weeks; and physical urticaria which are caused by something that stimulates the skin, for example cold, heat, pressure, sweating etc.

Neuropathic pain as used herein refers to pain caused by damage, injury or disease to nerve cells. The pain is commonly described by patients as a burning sensation and affected areas are often sensitive to touch. Typical symptoms of neuropathic pain includes pins and needles, difficulty sensing temperatures correctly, numbness, and extreme pain. Neuropathic pain may result from disorders of the peripheral nervous system or the central nervous system, for example fibromyalgia. Neuropathic pain may be thus broadly classified as peripheral neuropathic pain, central neuropathic pain or mixed (peripheral and central) neuropathic pain.

In one embodiment there is provided treatment of fibromyalgia.

Common treatments for neuropathic pain include anti-epileptics such as gabapentin and pregabalin; antidepressants such as amitriptyline and duloxetine; opioids such as a codeine, fentanyl and codeine; capsaicin cream; lidocaine patches; electrical nerve stimulations such as transcutaneous electrical nerve stimulation (TENS) and percutaneous electrical nerve stimulations (PENS) and alternative therapies such as acupuncture.

In one embodiment, the neuropathic pain is caused by or associated with neuropathy. The term neuropathy as used herein refers to disease or damage of the nerves.

In one embodiment the neuropathy is caused by one of the following: diabetes; trauma; autoimmune disorders such as Gullain-Barré syndrome, rheumatoid arthritis, Sjoren's syndrome; infections such as chickenpox, shingles, HIV, herpes, Epstein-Barr virus, West Nile virus and hepatitis C; kidney disorders; liver disorders; hypothyroidism; tumors (malignant or benign) that compress nerves; medications or poisons such as chemotherapy and radiation; vascular disorders; alcoholism; and inherited disorders such as Charcot-Marie Tooth disease, Fabry disease, and familial amyloidosis.

In one embodiment, the neuropathic pain is selected from the group comprising: peripheral neuropathic pain, for example peripheral neuropathic pain in the feet, legs, arms or hands; autonomic neuropathic pain; focal neuropathic pain, for example focal neuropathic pain in the head, hand, torso or leg, Bell's palsy; proximal neuropathic pain; neuropathic pain associated with diabetes; neuropathic pain associated with compression mononeuropathic neuropathy, such as Carpal tunnel syndrome; Phantom limb syndrome; trigeminal neuralgia; neuropathic pain associated with thoracic or lumbar radiculopathy; neuropathic pain associated with an infection, such as shingles, HIV or syphilis; sciatica; neuropathic pain associated with amputation; neuropathic pain associated with spine surgery; and neuropathic pain associated with multiple myeloma.

Interleukin-13 receptor (IL-13R) as used herein is a type I cytokine receptor, which binds to Interleukin-13. It consists of two subunits, encoded by IL13Rα1 and IL4R, respectively. These two genes encode the proteins IL-13Rα1 and IL-4Rα. These form a dimer with IL-13 binding to the IL-13Rα1 chain and IL-4Rα stabilises this interaction. Due to the presence of the IL4R subunit, IL13R can also instigate IL-4 signalling. In both cases this occurs via activation of the Janus kinase (JAK)/Signal Transducer and Activator of Transcription (STAT) pathway, resulting in phosphorylation of STAT6. Human IL-13Rα1 has the Uniprot number P3597.

IL-13Rα2, previously called IL-13R and IL-13Rα, is another receptor which is able to bind to IL-13. However, in contrast to IL-13Rα1, this protein binds IL-13 with high affinity, but it does not bind IL-4. Human IL-13Rα2 has the Uniprot number Q14627.

In one embodiment the anti-IL-13R antibody or binding fragment thereof of the present disclosure binds to IL-13Rα1. In one embodiment, the antibody or binding fragment thereof binds only to IL-13Rα1 and does not bind to IL-13Rα2.

In one embodiment CDRH1 is an amino acid sequence GYSFTSYYWIG (SEQ ID NO: 1).

In one embodiment CDRH2 is an amino acid sequence VIYPGDSYTR (SEQ ID NO: 2)

In one embodiment CDRH3 has the formula:

SEQ ID NO: 3
X₁ Pro Asn Trp Gly X₆ X₇ Asp X₉

X₁ denotes Phe, Met, Gln, Leu or Val
X₆ denotes Ser or Ala
X₇ denotes Phe, Leu, Ala or Met
X₉ denotes Tyr, Gln, Lys, Arg, Trp, His, Ala, Thr, Ser, Asn or Gly

In one embodiment the anti-IL13R antibody or binding fragment employed in the formulation of the present disclosure comprises a CDRH3 in dependently selected from SEQ ID NO: 4 to 30:

| SEQ ID NO: | CDR H3 SEQUENCE |
|---|---|
| 4 | FPNWGALDQ |
| 5 | VPNMGSLDT |
| 6 | FPNWGSMDA |
| 7 | FPNWGSLDH |
| 8 | MPNWGSFDY |
| 9 | MPNWGSFDT |
| 10 | MPNWGSLDH |
| 11 | MPNWGSFDS |
| 12 | MPNWGSLDT |
| 13 | MPNWGSLDA |
| 14 | MPNWGSLDN |
| 15 | MPNWGALDS |
| 15 | MPNWGSLDN |
| 17 | MPNWGSLDY |
| 18 | MPNWGSFDH |
| 19 | MPNWGSLDS |
| 20 | MPNWGSLDG |
| 21 | VPNWGSLDG |
| 22 | CARFPNWGSLDHWGQGTLVTVSSASIKG |
| 23 | CARMPNWGSLDHWGQGTLVTVSSASTKG |
| 24 | CARMPNWGSFDYWGQGTLVTVSSASIKG |
| 25 | VRMPNWGSLDHW |
| 26 | VRMPNWGSLDHWGQGTLVTVSSADIKG |
| 27 | ARMPNWGSLDHWGQGTLVTVSSASIKG |
| 28 | FPNWGSFDYWGQGTLVTVSSASIKG |
| 29 | VPNWGSLDA |
| 30 | FPNWGSFDY |

In one embodiment, the anti-IL13R antibody or binding fragment employed in the present disclosure comprises a VH CDR1 comprising an amino acid sequence as set forth in SEQ ID NO: 1, a VH CDR2 comprising an amino acid sequence as set forth in SEQ ID NO: 2, and a VH CDR3 comprising an amino acid sequence as set forth in SEQ ID NO: or 3.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 4, 5, 6, 7, 8, 9, 10, 11, 12, 13, 14, 15, 16, 17, 18, 19, 20, 21, 22, 23, 24, 25, 26, 27, 28, 29 or 30.

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment CDRL1 is an amino acid sequence RASQSISSSYLA SEQ ID NO: 31

In one embodiment CDRL2 is an amino acid sequence GASSRAT SEQ ID NO: 32

In one embodiment CDL3 has the formula:

SEQ ID NO: 33
Gln X₂X₃X₄X₅

X₂ denotes Gln, Arg, Met, Ser, Thr or Val.
X₃ denotes Tyr or Val.
X₄ denotes Glu, Ala, Gly or Ser.
X₅ denotes Thr, Ala or Ser.

In one embodiment the IL-13Rα1 antibody employed in the formulation of the present disclosure comprises a CDRL3 in dependently selected from SEQ ID NO: 34 to 47:

| SEQ ID NO: | CDR L3 SEQUENCE |
|---|---|
| 34 | QRYAT |
| 35 | QRYST |
| 36 | QMYST |
| 37 | QQVGT |
| 38 | QQVST |
| 39 | QQYST |
| 40 | QSYST |
| 41 | QQYAT |
| 42 | QQYSS |
| 43 | QTYST |
| 44 | QQYGS |
| 45 | QQYAS |
| 46 | QQYEA |
| 47 | QQYET |

In one embodiment, the anti-IL13R antibody or binding fragment employed in the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10.

In one embodiment the IL-13Rα1 antibody employed in the formulation of the present disclosure comprises a CDRL3 independently selected from a sequence comprising SEQ ID NO: 34 to 47.

In one embodiment CDRL3 comprising an amino acid sequence as set forth in SEQ ID NO: 33.

In one embodiment the VH region is independently selected from a sequence from the group comprising: SEQ ID NO: 48; SEQ ID NO: 49; SEQ ID NO: 50; SEQ ID NO: 51 and a sequence at least 95% identical to any one of the same.

SEQ ID NO: 51
Glu Val Gln Leu Val Gln Ser Gly Ala Glu
Val Lys Lys Pro Gly Glu Ser Leu Lys Ile
Ser Cys Lys Gly Ser Gly Tyr Ser Phe Thr
Ser Tyr Trp Ile Gly Trp Val Arg Gln Met
Pro Gly Lys Gly Leu Glu Trp Met Gly Val
Ile Tyr Pro Gly Asp Ser Tyr Thr Arg Tyr
Ser Pro Ser Phe Gln Gly Gln Val Thr Ile
Ser Ala Asp Lys Ser Ile Ser Thr Ala Tyr
Leu Gln Trp Ser Ser Leu Lys Ala Ser Asp
Thr Ala Met Tyr Tyr Cys Ala Arg Met Pro
Asn Trp Gly Ser Leu Asp His Trp Gly Gln
Gly Thr Leu Val Thr Val Ser Ser

In one embodiment the VL is independently selected from a sequence from the group comprising: SEQ ID NO: 52, SEQ ID NO: 53 and SEQ ID NO: 54 and a sequence at least 95% identical to any one of the same.

SEQ ID NO: 53
EIVLTQSPGTLSLSPGERATLSCRASQSISSSYLAWYQ
QKPGQAPRLLIYGASSRATGIPDRFSGSGSGTDFTLTI
SRLEPEDFAVYYCQQYASFGQGTKVEI*
(*K deleted in a post translational modification).

In one embodiment the VH sequence is SEQ ID NO: 48 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 49 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 50 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VH sequence is SEQ ID NO: 51 (or a sequence at least 95% identical thereto) and the VL sequence is SEQ ID NO: 52, SEQ ID NO: 53 or SEQ ID NO: 54 (or a sequence at least 95% identical to any one of the same).

In one embodiment the VL sequence is SEQ ID NO: 52 (or a sequence at least 95% identical thereto) and the VH sequence is SEQ ID NO: 48, SEQ ID NO: 49, SEQ ID NO: 50 or SEQ ID NO: 51. (or a sequence at least 95% identical to any one of the same).

In one embodiment the VL sequence is SEQ ID NO: 53 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VL sequence is SEQ ID NO: 54 (or a sequence at least 95% identical thereto) and the VH sequence is SEQ ID NO: 48, SEQ ID NO: 49, SEQ ID NO: 50 or SEQ ID NO: 51 (or a sequence at least 95% identical to any one of the same).

In one embodiment the VH sequence is SEQ ID NO: 51 (or a sequence at least 95% identical thereto) and the VL sequence is SEQ ID NO: 53 ((or a sequence at least 95% identical thereto).

Variable region as employed herein refers to the region in an antibody chain comprising the CDRs and a suitable framework.

In one embodiment the heavy chain comprises a sequence independently selected from the group comprising: SEQ ID NO: 55, SEQ ID NO: 56, SEQ ID NO: 57, SEQ ID NO: 58, SEQ ID NO: 59, SEQ ID NO: 60 and a sequence at least 95% identical to any one of the same.

In one embodiment the light chain is independently selected from a group comprising: SEQ ID NO: 61: SEQ ID NO: 62; SEQ ID NO: 63 and a sequence at least 95% identical to any one of the same.

In one embodiment the heavy chain is independently selected from SEQ ID NO: 55, 56, 57, 58, 59 and 60 (or a sequence at least 95% identical to any one of the same) and the light chain is independently selected from SEQ ID NO: 61 62 and 63 (or a sequence at least 95% identical to any one of the same).

In one embodiment the heavy chain is SEQ ID NO: 55 (or a sequence at least 95% identical thereto) and the light chain is independently selected from SEQ ID NO: 61 or 62 and 63 (or a sequence at least 95% identical to any one of the same).

In one embodiment the heavy chain is SEQ ID NO: 56 (or a sequence at least 95% identical thereto) and the light chain is independently selected from SEQ ID NO: 61 or 62 and 63 (or a sequence at least 95% identical to any one of the same).

In one embodiment the heavy chain is SEQ ID NO: 57 (or a sequence at least 95% identical thereto) and the light chain is independently selected from SEQ ID NO: 62 or 63 and 63 (or a sequence at least 95% identical to any one of the same).

In one embodiment the heavy chain is SEQ ID NO: 58 (or a sequence at least 95% identical thereto) and the light chain is independently selected from SEQ ID NO: 61 62 and 63 (or a sequence at least 95% identical to any one of the same).

In one embodiment the heavy chain is SEQ ID NO: 59 (or a sequence at least 95% identical thereto) and the light chain is independently selected from SEQ ID NO: 61 62 and 63 (or a sequence at least 95% identical to any one of the same).

In one embodiment the heavy chain is SEQ ID NO: 60 (or a sequence at least 95% identical thereto) and the light chain is independently selected from SEQ ID NO: 61 61 and 63 (or a sequence at least 95% identical to any one of the same).

In one embodiment the heavy chain is SEQ ID NO: 58 or 60 (or a sequence at least 95% identical to any one of the same) and a light chain with the sequence shown in SEQ ID NO: 61 (or a sequence at least 95% identical thereto).

In one embodiment the heavy chain is SEQ ID NO: 58 (or a sequence at least 95% identical to any one of the same) and a light chain with the sequence shown in SEQ ID NO: 61 (or a sequence at least 95% identical thereto).

In one embodiment the heavy chain is SEQ ID NO: 60 (or a sequence at least 95% identical to any one of the same) and a light chain with the sequence shown in SEQ ID NO: 61 (or a sequence at least 95% identical thereto).

In one embodiment, the anti-IL13R antibody of the present disclosure comprises a CDRH1 comprising an amino acid sequence as set forth in SEQ ID NO: 1, a CDRH2 comprising an amino acid sequence as set forth in SEQ ID NO: 2, and a CDRH3 comprising an amino acid sequence as set forth in SEQ ID NO: 10, a CDRL1 comprising an amino acid sequence SEQ ID NO: 31, a CDRL2 comprising an amino acid sequence SEQ ID NO: 32, and a CDRL3 comprising an amino acid sequence as set forth in SEQ ID NO: 45.

In one embodiment, the anti-IL13R antibody is eblasakimab (previously known as ASLAN004).

Derived from as employed herein refers to the fact that the sequence employed or a sequence highly similar to the sequence employed was obtained from the original genetic material, such as the light or heavy chain of an antibody.

“At least 95% identical” as employed herein is intended to refer to an amino acid sequence which over its full length is 95% identical or more to a reference sequence, such as 96, 97, 98 or 99% identical. Software programmes can be employed to calculate percentage identity.

Any discussion of a protein, antibody or amino acid sequence herein will be understood to include any variants of the protein, antibody or amino acid sequence produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” (C-terminal lysine residues of encoded antibodies are often removed during the manufacturing process) and/or have part or all of a signal sequence incompletely processed and, as a consequence, remain at the terminus of the antibody. It is understood that an antibody comprising a particular amino acid sequence or binding fragment thereof may be a heterogeneous mixture of the stated or encoded sequence and/or variants of that stated or encoded sequence or binding fragment thereof.

In one embodiment the present disclosure extends to a sequence explicitly disclosed herein where the C-terminal lysine has been cleaved.

In one embodiment an antibody or binding fragment thereof, employed in a formulation of the present disclosure is humanised.

Humanised (which include CDR-grafted antibodies) as employed herein refers to molecules having one or more complementarity determining regions (CDRs) from a non-human species and a framework region from a human immunoglobulin molecule (see, for example U.S. Pat. No. 5,585,089; W091/09967). It will be appreciated that it may only be necessary to transfer the specificity determining residues of the CDRs rather than the entire CDR (see for example, Kashmiri et al 2005, Methods, 36, 25-34). Humanised antibodies may optionally further comprise one or more framework residues derived from the non-human species from which the CDRs were derived. For a review, see Vaughan et al, Nature Biotechnology, 16, 535-539, 1998.

When the CDRs or specificity determining residues are grafted, any appropriate acceptor variable region framework sequence may be used having regard to the class/type of the donor antibody from which the CDRs are derived, including mouse, primate and human framework regions. Examples of human frameworks which can be used in the present invention are KOL, NEWM, REI, EU, TUR, TEI, LAY and POM (Kabat et al). For example, KOL and NEWM can be used for the heavy chain, REI can be used for the light chain and EU, LAY and POM can be used for both the heavy chain and the light chain. Alternatively, human germline sequences may be used; these are available at: http://vbase.mrc-cpe.cam.ac.uk/

In a humanised antibody employed in the present invention, the acceptor heavy and light chains do not necessarily need to be derived from the same antibody and may, if desired, comprise composite chains having framework regions derived from different chains.

The framework regions need not have exactly the same sequence as those of the acceptor antibody. For instance, unusual residues may be changed to more frequently-occurring residues for that acceptor chain class or type. Alternatively, selected residues in the acceptor framework regions may be changed so that they correspond to the residue found at the same position in the donor antibody (see Reichmann et al., 1998, Nature, 332, 323-324). Such changes should be kept to the minimum necessary to recover the affinity of the donor antibody. A protocol for selecting residues in the acceptor framework regions which may need to be changed is set forth in W091/09967.

In one embodiment the anti-IL13R antibodies of the present disclosure are fully human, in particular one or more of the variable domains are fully human.

Fully human molecules are those in which the variable regions and the constant regions (where present) of both the heavy and the light chains are all of human origin, or substantially identical to sequences of human origin, not necessarily from the same antibody. Examples of fully human antibodies may include antibodies produced, for example by the phage display methods described above and antibodies produced by mice in which the murine immunoglobulin variable and optionally the constant region genes have been replaced by their human counterparts e.g. as described in general terms in EP0546073, U.S. Pat. Nos. 5,545,806, 5,569,825, 5,625,126, 5,633,425,5,661,016, 5,770,429, EP0438474 and EP0463151.

Thus, the presently disclosed anti-IL13R antibody may comprise one or more constant regions, such as a naturally occurring constant domain or a derivate of a naturally occurring domain. A derivative of a naturally occurring domain as employed herein is intended to refer to where one, two, three, four or five amino acids in a naturally occurring sequence have been replaced or deleted, for example to optimize the properties of the domain such as by eliminating undesirable properties but wherein the characterizing feature(s) of the domain is/are retained.

If desired an antibody for use in the present invention may be conjugated to one or more effector molecule(s). It will be appreciated that the effector molecule may comprise a single effector molecule or two or more such molecules so linked as to form a single moiety that can be attached to the antibodies of the present invention. Where it is desired to obtain an antibody fragment linked to an effector molecule, this may be prepared by standard chemical or recombinant DNA procedures in which the antibody fragment is linked either directly or indirectly including via a coupling agent to the effector molecule. Techniques for conjugating such effector molecules to antibodies are well known in the art (see, Hellstrom et al, Controlled Drug Delivery, 2nd Ed., Robinson et al, eds., 1987, pp. 623-53; Thorpe et al, 1982, Immunol. Rev., 62:119-58 and Dubowchik et al, 1999, Pharmacology and Therapeutics, 83, 67-123). Particular chemical procedures include, for example, those described in W093/06231, W092/22583, W089/00195, W089/01476 and WO03/031581. Alternatively, where the effector molecule is a protein or polypeptide the linkage may be achieved using recombinant DNA procedures, for example as described in W086/01533 and EP0392745.

The term effector molecule as used herein includes, for example, biologically active proteins, for example enzymes, other antibody or antibody fragments, synthetic or naturally occurring polymers, nucleic acids and fragments thereof eg DNA, RNA and fragments thereof, radionuclides, particularly radioiodide, radioisotopes, chelated metals, nanoparticles and reporter groups such as fluorescent compounds or compounds which may be detected by NMR or ESR spectroscopy.

Other effector molecules may include detectable substances useful for example in diagnosis. Examples of detectable substances include various enzymes, prosthetic groups, fluorescent materials, luminescent materials, bioluminescent materials, radioactive nuclides, positron emitting metals (for use in positron emission tomography), and nonradioactive paramagnetic metal ions. See generally U.S. Pat. No. 4,741,900 for metal ions which can be conjugated to antibodies for use as diagnostics. Suitable enzymes include horseradish peroxidase, alkaline phosphatase, beta-galactosidase, or acetylcholinesterase; suitable prosthetic groups include streptavidin, avidin and biotin; suitable fluorescent materials include umbelliferone, fluorescein, fluorescein isothiocyanate, rhodamine, dichlorotriazinylamine fluorescein, dansyl chloride and phycoerythrin; suitable luminescent materials include luminol; suitable bioluminescent materials include luciferase, luciferin, and aequorin; and suitable radioactive nuclides include 125I, 131I, 111In and 99Tc.

In another example the effector molecule may increase the half-life of the antibody in vivo, and/or reduce immunogenicity of the antibody and/or enhance the delivery of an antibody across an epithelial barrier to the immune system. Examples of suitable effector molecules of this type include polymers, albumin, albumin binding proteins or albumin binding compounds such as those described in WO05/117984. Where the effector molecule is a polymer it may, in general, be a synthetic or a naturally occurring polymer, for example an optionally substituted straight or branched chain polyalkylene, polyalkenylene or polyoxyalkylene polymer or a branched or unbranched polysaccharide, e.g. a homo- or hetero-polysaccharide.

Specific optional substituents which may be present on the above-mentioned synthetic polymers include one or more hydroxy, methyl or methoxy groups.

Specific examples of synthetic polymers include optionally substituted straight or branched chain poly(ethyleneglycol), poly(propyleneglycol) poly(vinylalcohol) or derivatives thereof, especially optionally substituted poly(ethyleneglycol) such as methoxypoly(ethyleneglycol) or derivatives thereof.

Specific naturally occurring polymers include lactose, amylose, dextran, glycogen or derivatives thereof.

“Derivatives” as used herein is intended to include reactive derivatives, for example thiol-selective reactive groups such as maleimides and the like. The reactive group may be linked directly or through a linker segment to the polymer. It will be appreciated that the residue of such a group will in some instances form part of the product as the linking group between the antibody fragment and the polymer.

Suitable polymers include a polyalkylene polymer, such as a poly(ethyleneglycol) or, especially, a methoxypoly(ethyleneglycol) or a derivative thereof, and especially with a molecular weight in the range from about 15000Da to about 40000Da.

In one example antibodies for use in the present invention are attached to poly (ethyleneglycol) (PEG) moieties. In one particular example the antibody is an antibody fragment and the PEG molecules may be attached through any available amino acid side-chain or terminal amino acid functional group located in the antibody fragment, for example any free amino, imino, thiol, hydroxyl or carboxyl group. Such amino acids may occur naturally in the antibody fragment or may be engineered into the fragment using recombinant DNA methods (see for example U.S. Pat. Nos. 5,219,996; 5,667,425; WO98/25971, WO2008/038024). In one example the antibody molecule of the present invention is a modified Fab fragment wherein the modification is the addition to the C-terminal end of its heavy chain one or more amino acids to allow the attachment of an effector molecule. Suitably, the additional amino acids form a modified hinge region containing one or more cysteine residues to which the effector molecule may be attached. Multiple sites can be used to attach two or more PEG molecules.

In patients with cancer, such as breast cancer, cancer related lymphedema (BCRL), the formulation of the present disclosure may prevent lymphedema-associated effects, such as fibrosis, hyperkeratosis, the deposition of fibroadipose tissue, fluid accumulation, limb swelling, reduction of skin elasticity, and pain. By reducing the excess volume, said formulation may improve lymphatic and, for example limb functions.

The development of lymphedema after lymphatic injury is associated with tissue inflammation, the infiltration of CD4-positive cells and their differentiation to the type 2 helper T-cell (Th2) phenotype. Th2 cells produce IL-4 and IL-13 that play a key role in the development of lymphedema-associated symptoms as well as other Th2-mediated diseases.

In one embodiment the formulation herein is administered in combination with another therapy.

“In combination” as employed herein is intended to encompass where the anti-IL13R antibody is administered before, concurrently with another therapy or after another therapy, as the same or different formulations. Thus, combination is where the pharmacological effect of a first therapy exists at the same as the existence of a pharmacological effect of second therapy in the body and/or the two therapies are part of treatment plan designed to be employed together.

Therapeutic dose as employed herein refers to the amount of the anti-IL13R antibody, such as eblasakimab (ASLAN004) that is suitable for achieving the intended therapeutic effect when employed in a suitable treatment regimen, for example ameliorates symptoms or conditions of a disease, in particular without eliciting dose limiting side effects. Suitable therapeutic doses are generally a balance between therapeutic effect and tolerable toxicity, for example where the side-effect and toxicity are tolerable given the benefit achieved by the therapy.

In one embodiment a formulation according to the present disclosure (including a formulation comprising same) is administered monthly, for example in a treatment cycle or as maintenance therapy.

Unit dose as used herein generally refers to a product comprising the amount of anti-IL13R antibody or binding fragment thereof of the present disclosure that is administered in a single dose including any overage.

A unit dose of the presently claimed anti-IL13R antibody or antigen binding fragment thereof may refer to the marketed form of the product, such as a formulation of the anti-IL13R antibody or binding fragment thereof, wherein the product is apportioned into the amount of anti-IL13R antibody that is required for a single dose. Thus, the manufacturer is able to determine and control the exact amount of anti-13R antibody or binding fragment thereof to be included in each unit dose.

The product may be in various forms, familiar to the skilled addressee, such as vials, ampoules, infusion bags or a device (including an auto-injection device).

The exact amount as employed herein refers to the amount to be administer as a dose to the patient and any overage.

In one embodiment, the unit dose or unit doses are for use according to a method of the present disclosure.

In the context of this specification “comprising” is to be interpreted as “including”. Embodiments of the invention comprising certain features/elements are also intended to extend to alternative embodiments “consisting” or “consisting essentially” of the relevant elements/features. Where technically appropriate, embodiments of the invention may be combined.

Technical references such as patents and applications are incorporated herein by reference. Any embodiments specifically and explicitly recited herein may form the basis of a disclaimer either alone or in combination with one or more further embodiments.

The background section of this specification contains relevant technical information and may be used as basis for amendment.

Subject headings herein are employed to divide the document into sections and are not intended to be used to construe the meaning of the disclosure provided herein.

The present invention is further described by way of illustration only in the following examples.

### BRIEF DESCRIPTION OF FIGURES

FIG. 1A Table showing demographics of full analysis set

FIG. 1B Table showing baseline disease characteristics of full analysis set

FIG. 1C Table showing baseline disease characteristics of Evaluable for Efficacy set (EES)

FIG. 2A Table showing % change from baseline in EASI score at Day 57 for EES.

FIG. 2B Graph showing % change from baseline in EASI score at Day 57 for EES (ASLAN004 200 mg, 400 mg and 600 mg)

FIG. 2C Graph showing % change from baseline in EASI score at Day 57 for EES (ASLAN004 low dose and high doses)

FIG. 3A Table showing % change from baseline in EASI score at Day 29 for EES

FIG. 3B Graph showing % change from baseline in EASI score at Day 29 for EES (ASLAN004 200 mg, 400 mg and 600 mg)

FIG. 4A Graph showing % change in EASI score over time for EES (ASLAN004 200 mg, 400 mg and 600 mg)

FIG. 4B Graph showing % change in EASI score over time for EES (ASLAN004 low and high dose)

FIG. 5A Graph showing % change in baseline in EASI score over time for EES individual patients (Placebo)

FIG. 5B Graph showing % change in baseline in EASI score over time for EES individual patients (ASLAN004 200 mg)

FIG. 5C Graph showing % change in baseline in EASI score over time for EES individual patients (ASLAN004 400 mg)

FIG. 5D Graph showing % change in baseline in EASI score over time for EES (ASLAN004 600 mg)

FIG. 6A Table showing Day 57 sensitivity analysis in the modified intention to treat (mITT)

FIG. 6B Graph showing Day 57 sensitivity analysis in the mITT (ASLAN004 200 mg, 400 mg and 600 mg)

FIG. 6C Graph showing Day 57 sensitivity analysis in the mITT (ASLAN004 low and high dose)

FIG. 7A Summary table showing EASI 50, EASI 75, EASI 90 at Day 57 for EES

FIG. 7B Graph showing EASI 50 at Day 57 for EES (ASLAN004 200 mg, 400 mg and 600 mg)

FIG. 7C Graph showing EASI 50 at Day 57 for EES (ASLAN004 low and high dose)

FIG. 7D Graph showing EASI 75 at Day 57 for EES (ASLAN004 200 mg, 400 mg and 600 mg)

FIG. 7E Graph showing EASI 75 at Day 57 for EES (ASLAN004 low and high dose)

FIG. 7F Graph showing EASI 90 at Day 57 for EES (ASLAN004 200 mg, 400 mg and 600 mg)

FIG. 7G Graph showing EASI 90 at Day 57 for EES (ASLAN004 low and high dose)

FIG. 8A Graph showing proportion of patients achieving EASI 50

FIG. 8B Graph showing proportion of patients achieving EASI 75

FIG. 8C Graph showing proportion of patients achieving EASI 90

FIG. 9 Summary table showing proportion of patients achieving EAS 150, 75, 90 for EES and mITT

FIG. 10A Summary table showing proportion of patients with IGA score of 0 or 1 at Day 57 for ESS

FIG. 10B Graph showing proportion of patients with IGA score of 0 or 1 at Day 57 for ESS

FIG. 10C Graph showing proportion of patients with IGA score of 0 or 1 over time for ESS

FIG. 11 Table showing baseline TARC and IgE levels of patients

FIG. 12A Graph showing average % change from baseline TARC (ASLAN004 200 mg and 400 mg)

FIG. 12B Graph showing average % change from baseline TARC (ASLAN004 400 mg and placebo)

FIG. 12C Graph showing % change from baseline TARC for individual patients (ASLAN004 400 mg)

FIG. 13A Graph showing IgE % change from baseline for ASLAN004 200 mg and 400 mg

FIG. 13B Graph showing average IgE % change from baseline for ASLAN004 200 mg and 400 mg

FIG. 13C Graph showing IgE % change from baseline for individual patients for placebo

FIG. 13D Graph showing IgE % change from baseline for individual patients for ASLAN004 200 mg

FIG. 13E Graph showing IgE % change from baseline for individual patients for ASLAN004 400 mg

FIG. 14 Graph showing comparison between ASLAN004 exposure, EASI, TARC and IgE over time for patients receiving ASLAN004 400 mg

FIG. 15 Table showing comparison between ASLAN004 and Duplilumab

FIG. 16 Graph showing worst itch (peak P-NRS) improvement over time-time course of the response in the modified intent to treat (mITT) population. Statistical comparison is at week 8 (day 57) only.

FIG. 17 Schematic showing test protocol for Example 5.

FIG. 18A Graph showing change in fluorescent intensity ratio (ΔF/F₀) for BAM8-22 with IL-4 pre-stimulation

FIG. 18B Graph showing change in fluorescent intensity ratio (ΔF/F₀) for BAM8-22 with IL-13 pre-stimulation

FIG. 18C Graph showing change in fluorescent intensity ratio (ΔF/F₀) for BAM8-22 with IL-4 and IL-13 co-stimulation

FIG. 19A Graph showing BAM8-22 response for human sensory neurons pre-stimulated with IL-4, with or without Eblasakimab treatment

FIG. 19B Graph showing BAM8-22 response for human sensory neurons pre-stimulated with IL-13, with or without Eblasakimab treatment

FIG. 19C Graph showing BAM8-22 response for human sensory neurons pre-stimulated with IL-4 and IL-13, with or without Eblasakimab treatment

FIG. 20A Graph showing comparison in BAM8-22 response for human sensory neurons pre-stimulated with IL-4, IL-13 or both IL-4 and IL-13 with Eblasakimab treatment

FIG. 20B Summary graph showing comparison in BAM8-22 response for human sensory neurons pre-stimulated with all cytokines, with or without Eblasakimab treatment

FIG. 21A Graph showing PAMP-20 response for human dorsal root ganglias (hDRG) pre-stimulated with IL-4 with or without Eblasakimab treatment

FIG. 21B Graph showing PAMP-20 response for human dorsal root ganglias (hDRG) pre-stimulated with IL-13 with or without Eblasakimab treatment

FIG. 22A Graph showing neuronal spontaneous activity (SA) for human dorsal root ganglias (hDRG) treated with vehicle and in combination with inflammatory soup (IS)

FIG. 22B Graph showing neuronal spontaneous activity (SA) for human dorsal root ganglias (hDRG) treated with vehicle+Eblasakimab and in combination with inflammatory soup (IS)

FIG. 22C Graph showing neuronal spontaneous activity (SA) for human dorsal root ganglias (hDRG) treated with IL-4 and in combination with inflammatory soup (IS)

FIG. 22D Graph showing neuronal spontaneous activity (SA) for human dorsal root ganglias (hDRG) treated with IL-4+Eblasakimab and in combination with inflammatory soup (IS)

FIG. 23 Summary graph showing neuronal spontaneous activity (SA) for human dorsal root ganglias (hDRG) treated with various combinations of IL-4, IL-13, eblaskimab and inflammatory soup (IS). Values shown are average from 3 donors. Data Y-axis=% spontaneous activity, +inflammation=+inflammatory soup (IS).

FIG. 24 Flow chart showing number of subjects in each test group

FIG. 25 Table showing baseline demographics and disease characteristics of Intention-to-treat (ITT), modified Intention-to-treat (mITT) and Excluded site groups

FIG. 26 Table showing adverse events (AEs) for mITT vs Excluded site groups

FIG. 27A Graph showing improvement in (median) worst itch over time for mITT group

FIG. 27B Graph showing improvement in (median) worst itch at week 8 for mITT group

FIG. 28A Graph showing improvement in (median) average itch over time for mITT group

FIG. 28B Graph showing improvement in (median) average itch at week 8 for mITT group

FIG. 29 Graph showing improvement in POEM score change from baseline (CFBL) over time

FIG. 30 Graph showing 2-point improvement in sleep disturbance (SD) score at week 8 for miTT group

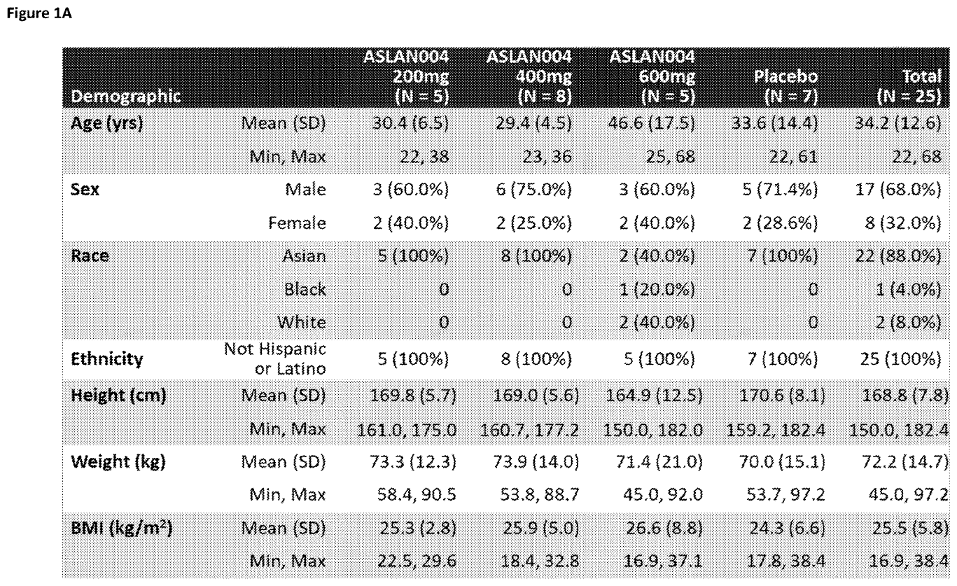

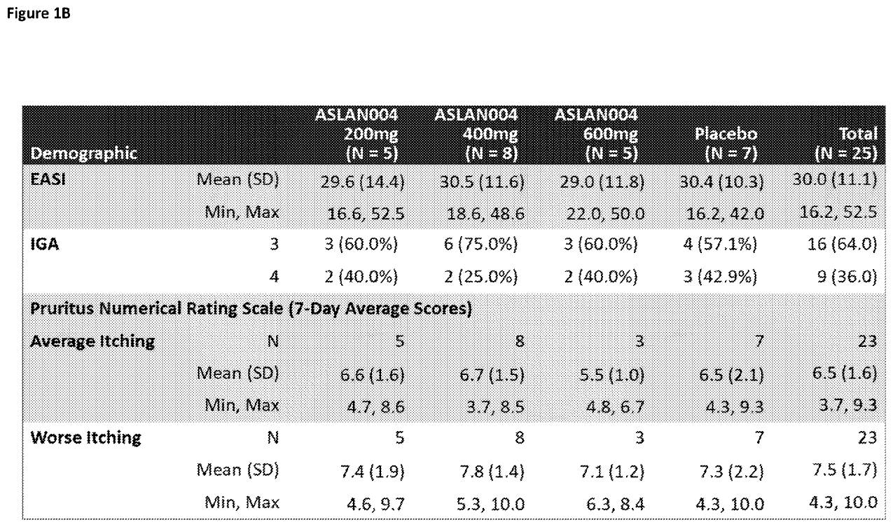

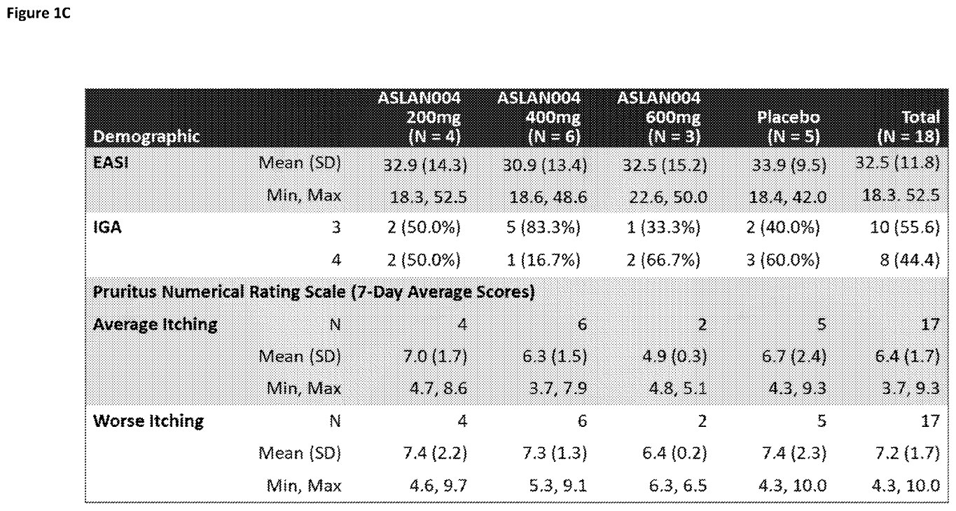

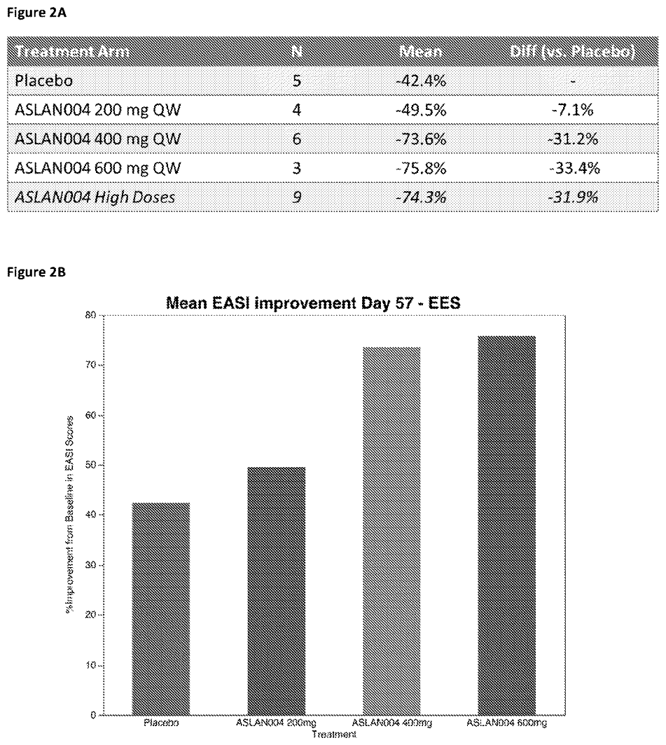

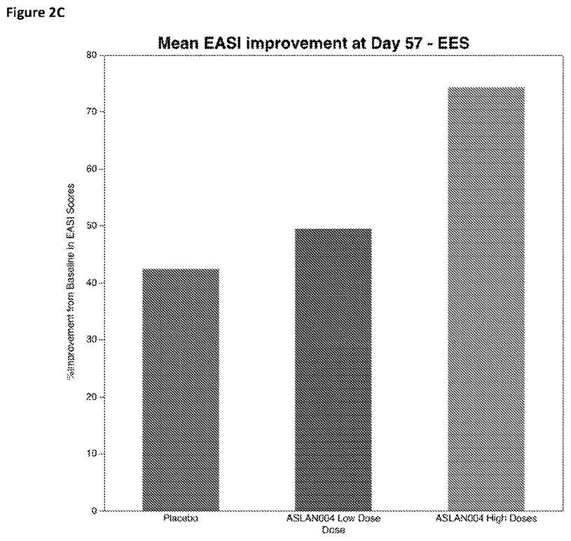

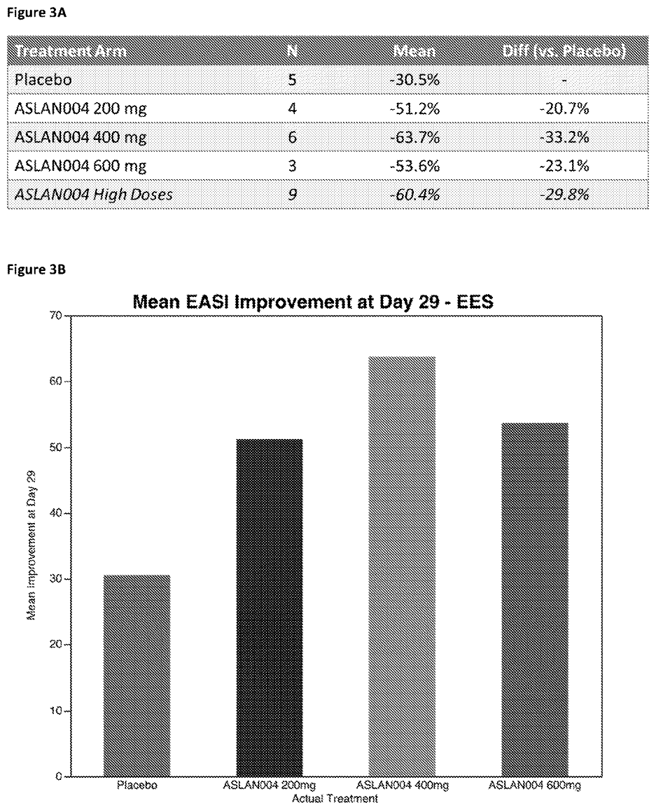

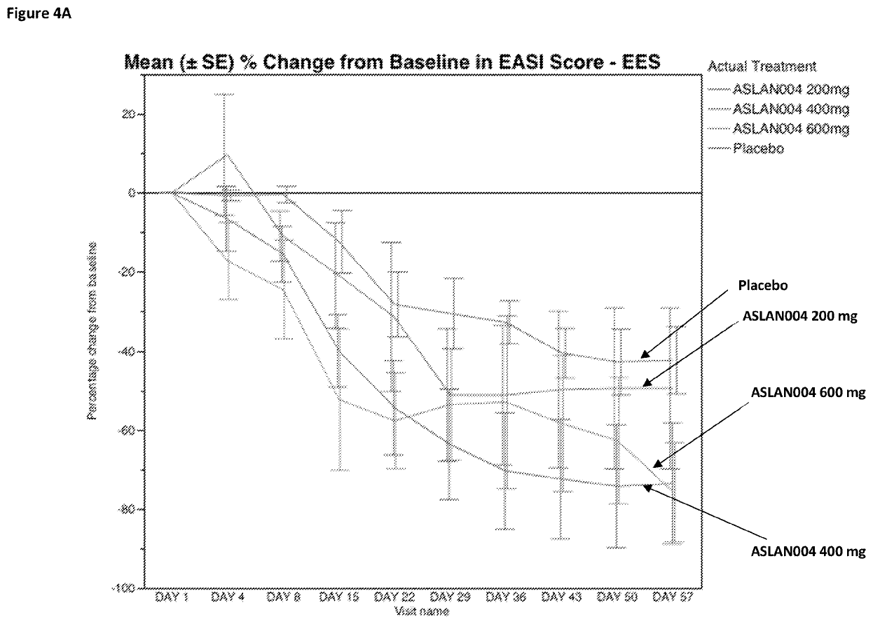

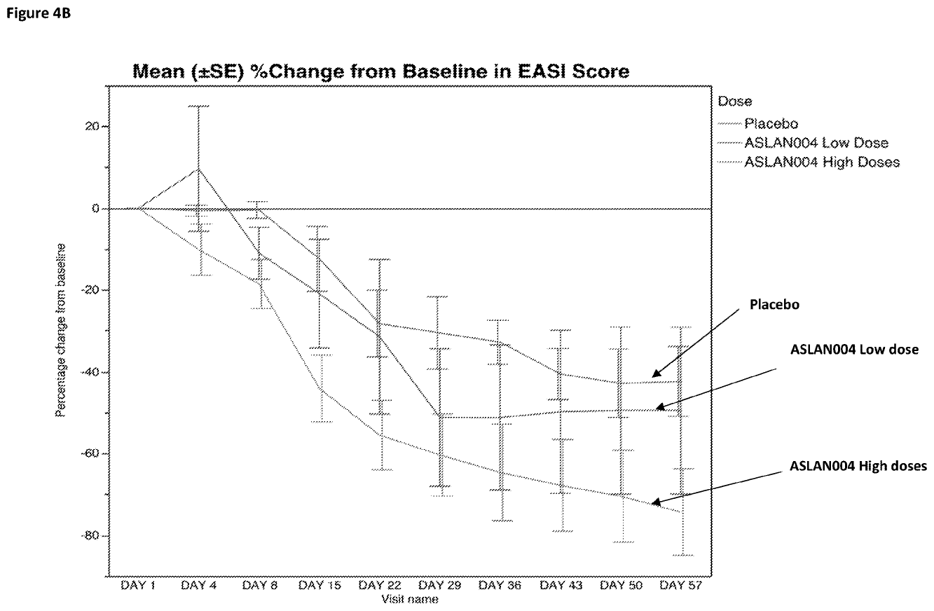

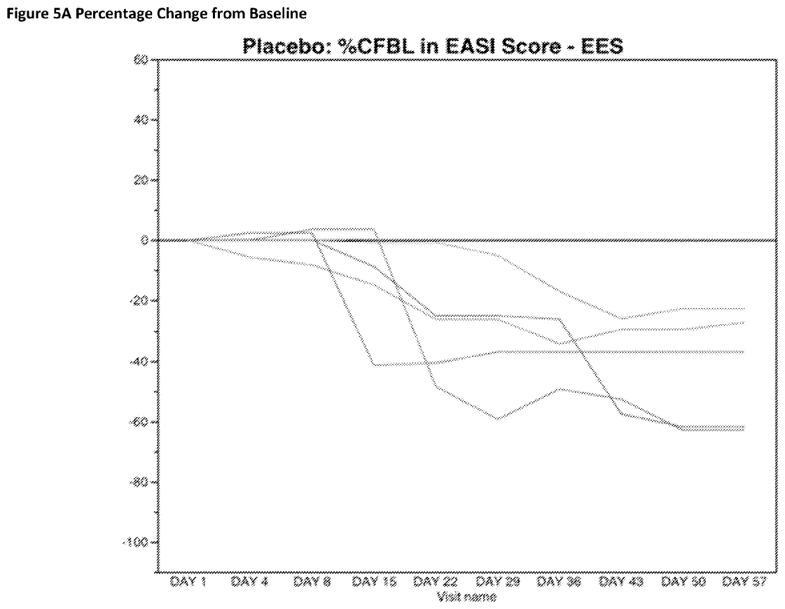

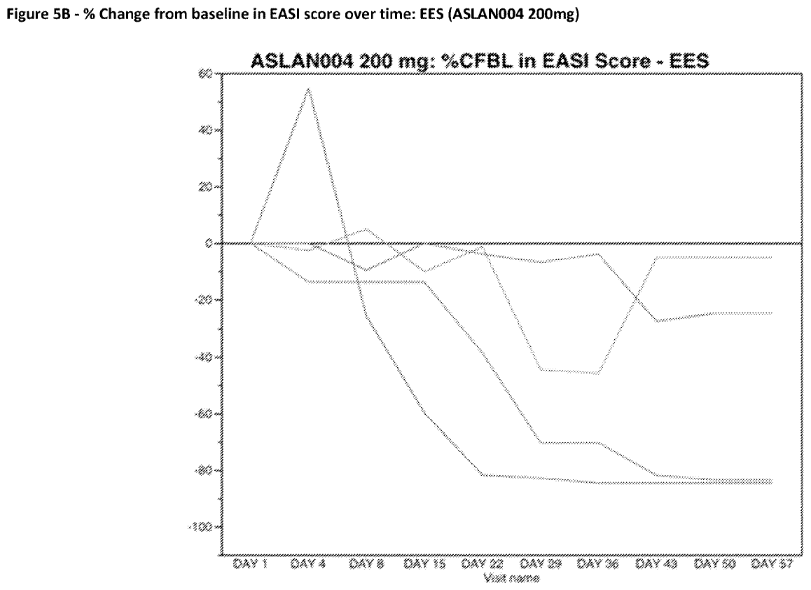

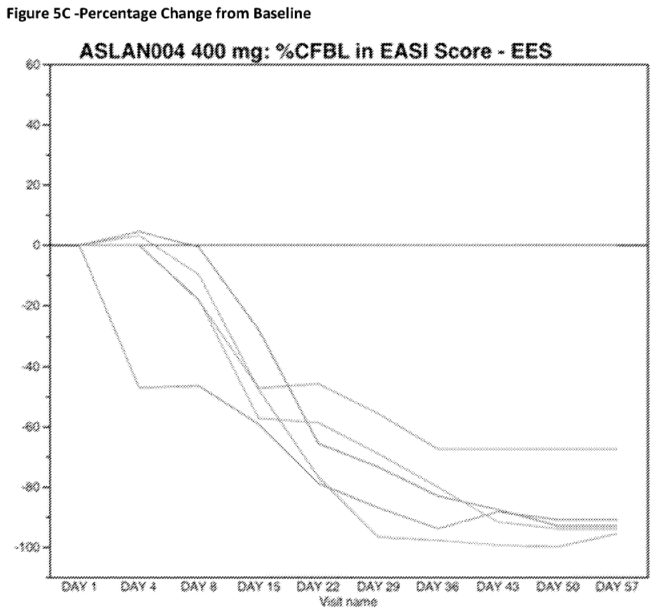

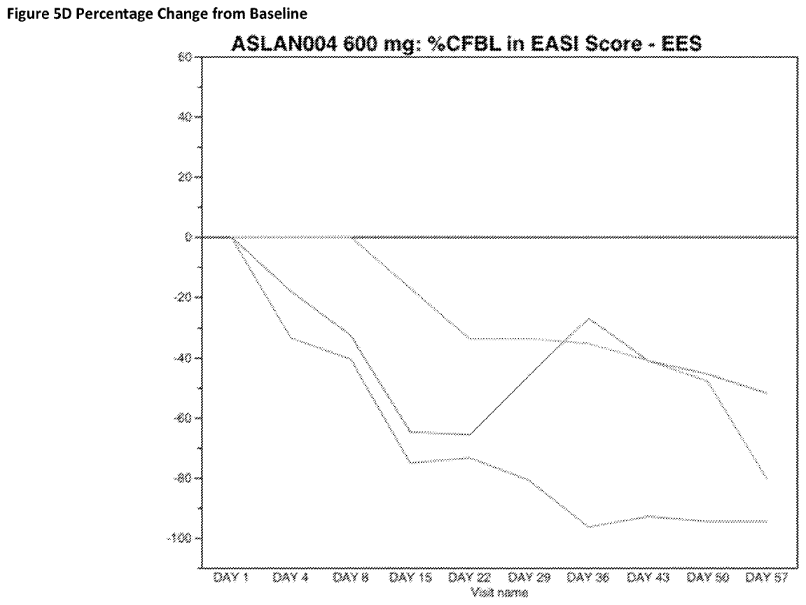

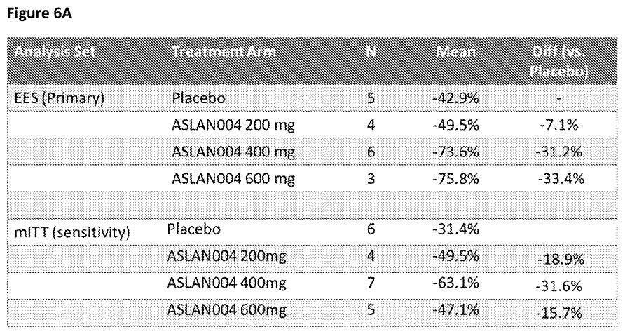

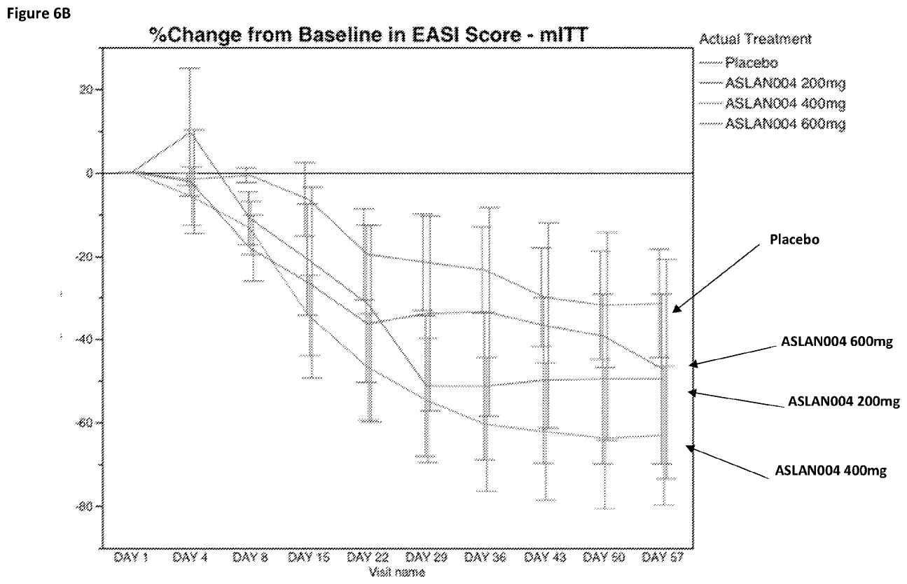

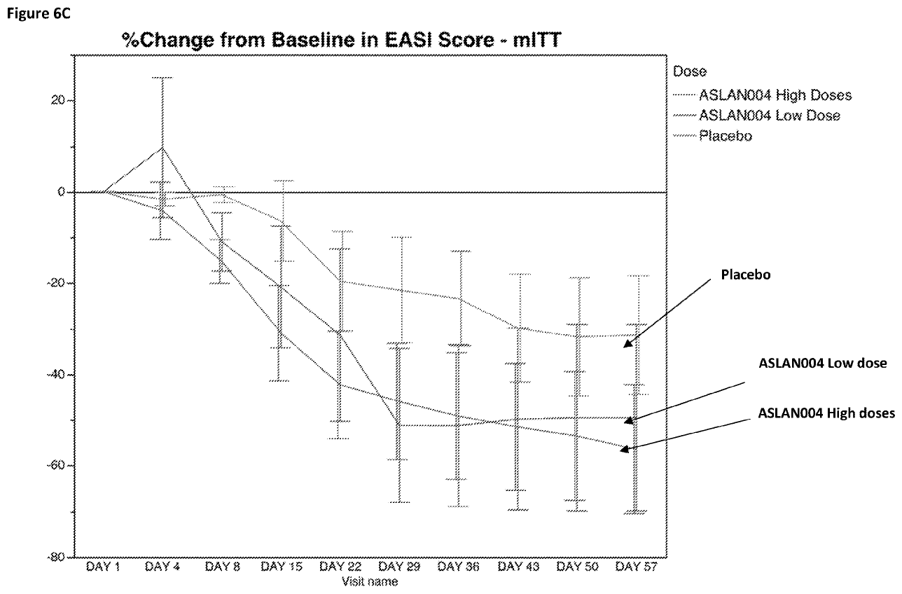

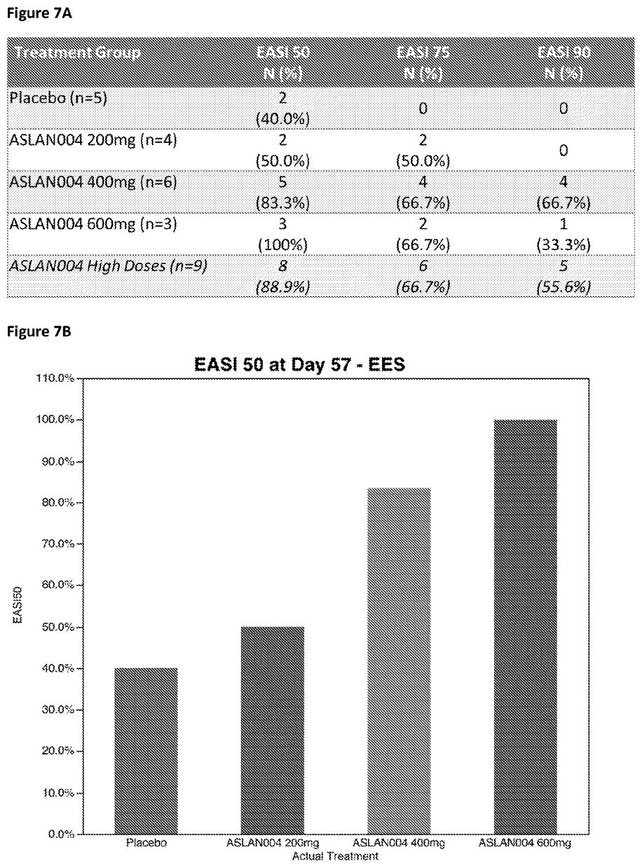

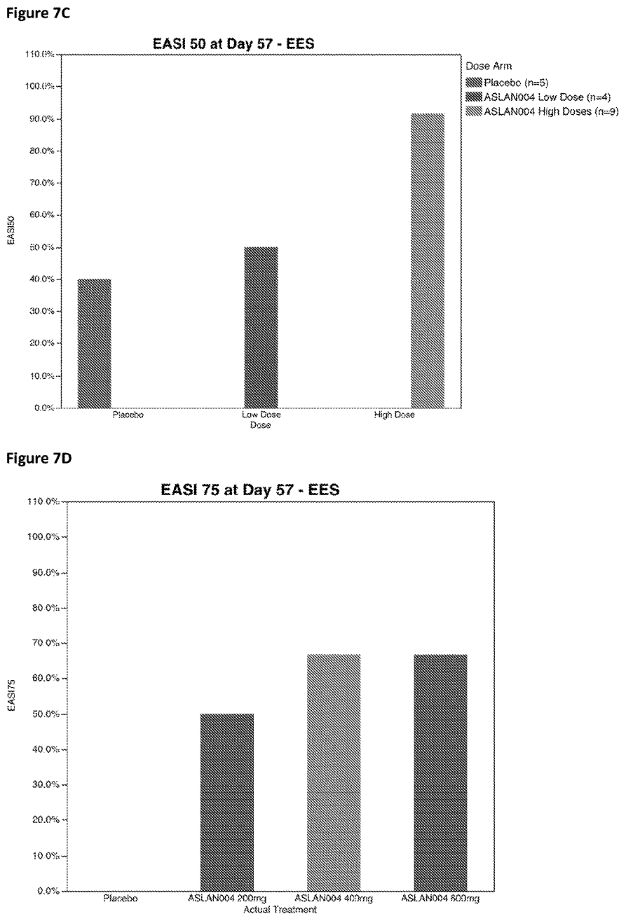

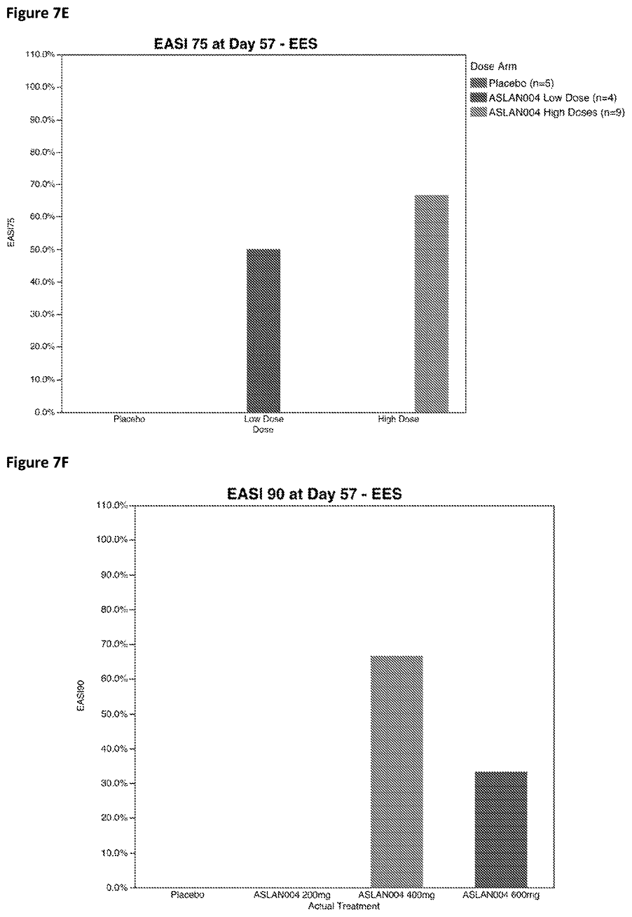

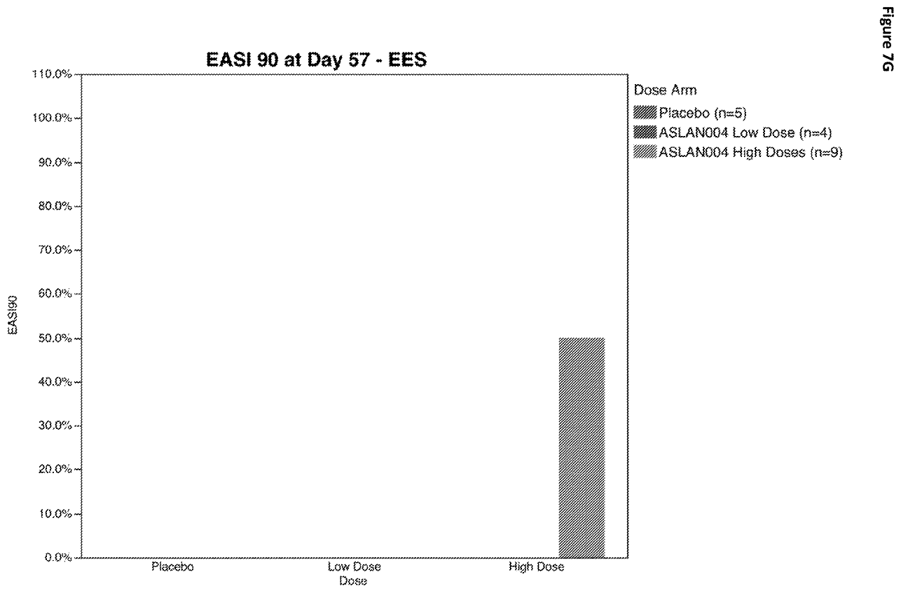

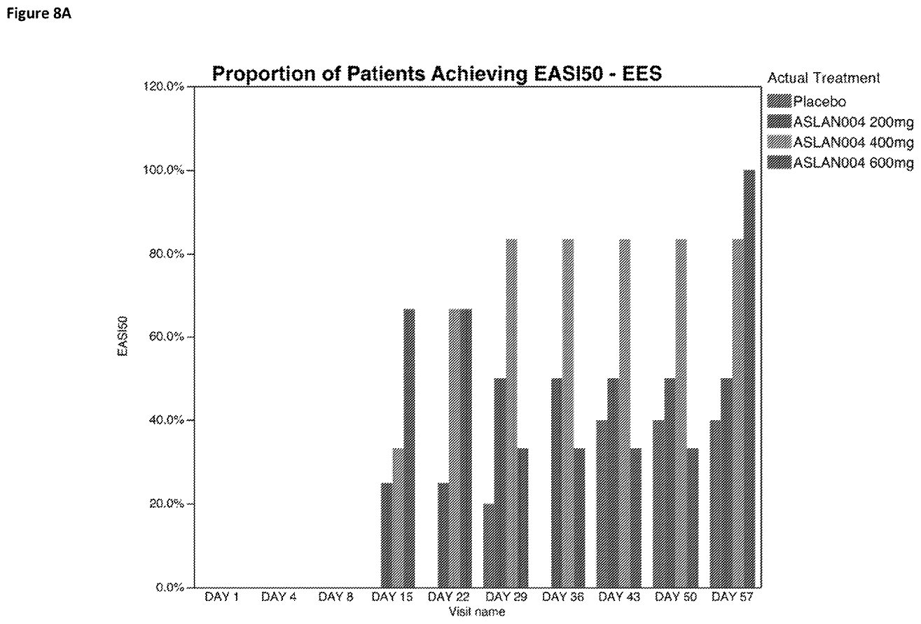

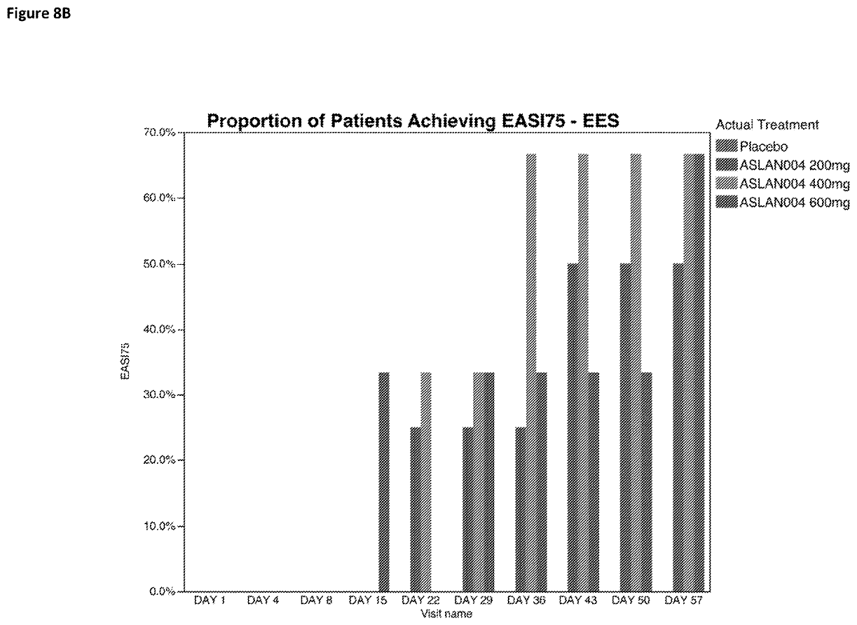

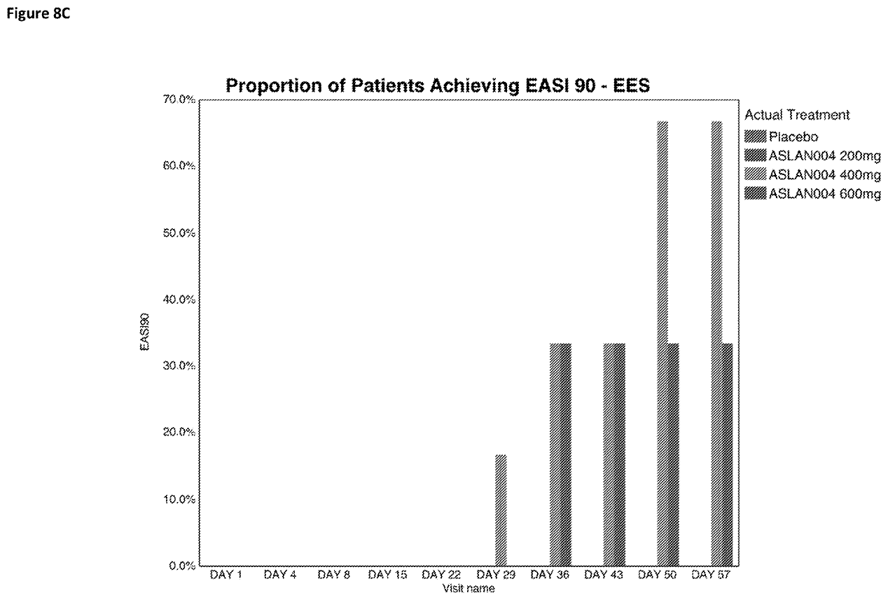

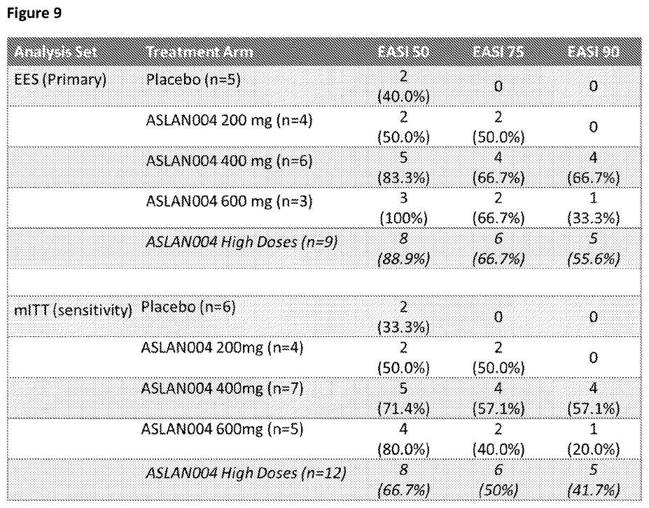

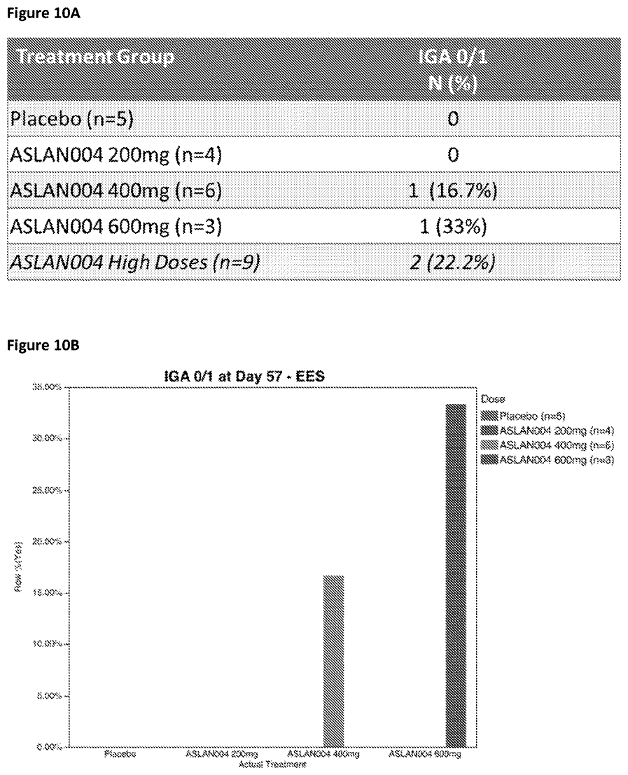

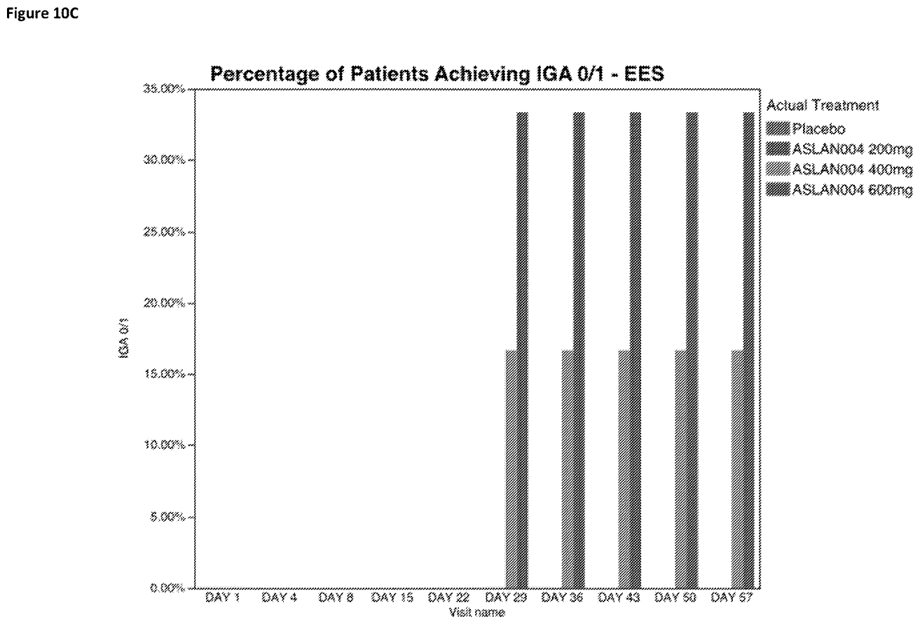

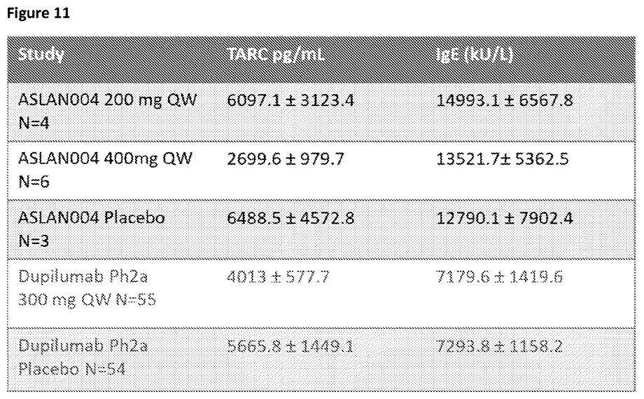

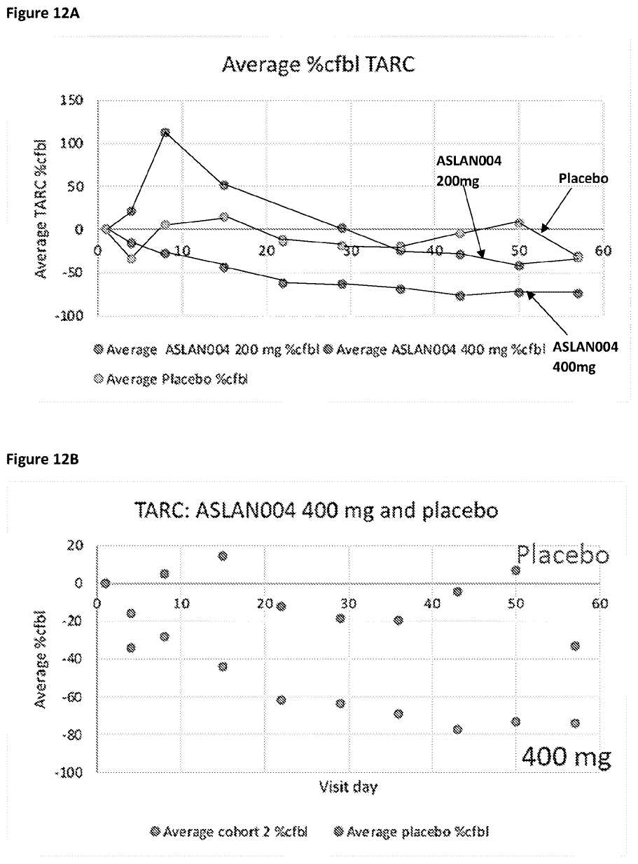

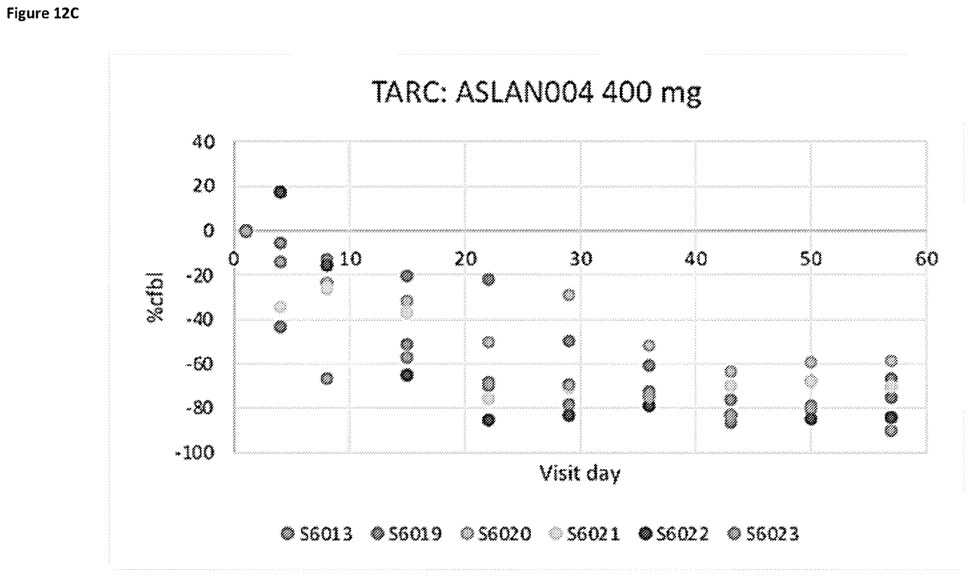

### EXAMPLES

### Example 1 Study Protocol (Initial MAD Escalation)

Patients enrolled in ascending dose cohorts of ASLAN004 (SEQ ID NO: 51, 53 and 59 herein): 200 mg, 400 mg, 600 mg. Initially the doses were given QW. Within each cohort, patients were randomized in a 3:1 ratio of ASLAN004:Placebo

TABLE 1
Baseline TARC and IgE

| Study | TARC pg/mL | IgE (KU/L) |
|---|---|---|
| ASLAN004 200 | 6097.1 + 3123.4 | 14993.1 + 6567.8 |
| MG QW N = 4 | | |
| ASLAN004 400 | 2699.6 + 979.7 | 13521.7 + 5362.5 |
| MG QW N = 6 | | |
| ASLAN004 | 6488.5 + 4572.8 | 12790.1 + 7902.4 |
| Placebo N = 3 | | |
| Dupilumab Ph2a | 4013 + 577.7 | 7179.6 + 1419.6 |
| 300 mg QW N = 55 | | |
| Dupilumab Ph2a | 5665.8 + 1449.1 | 7293.8 + 1158.2 |
| Placebo N = 54 | | |

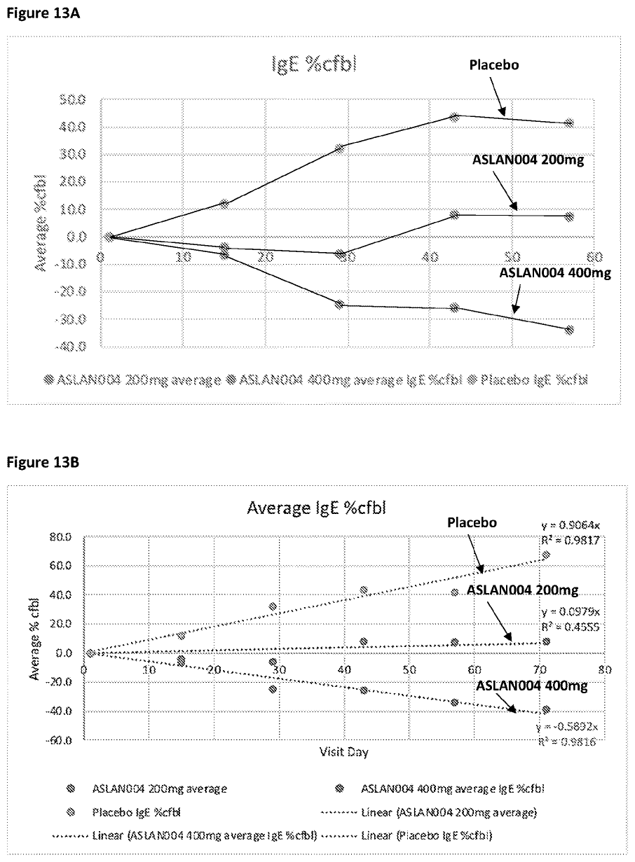

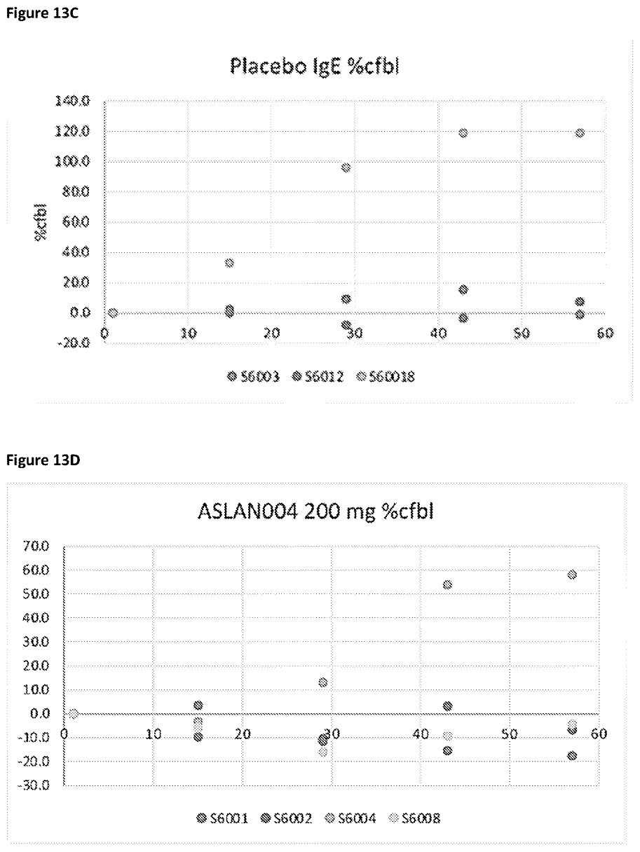

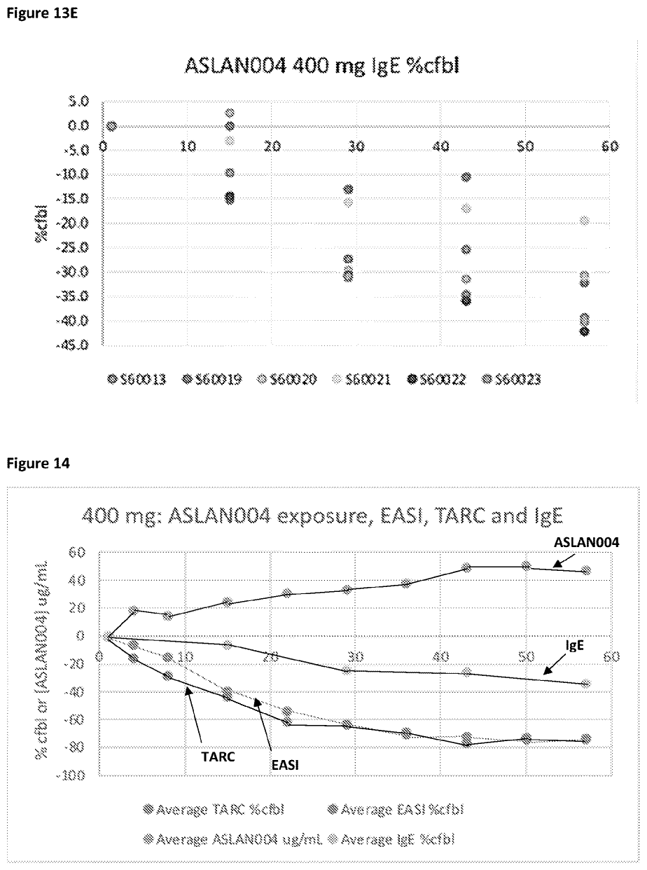

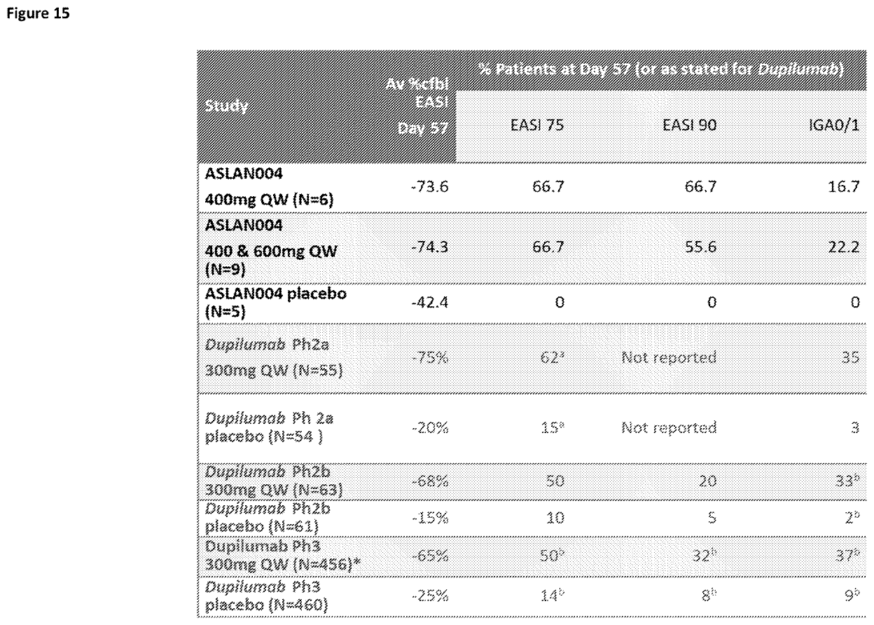

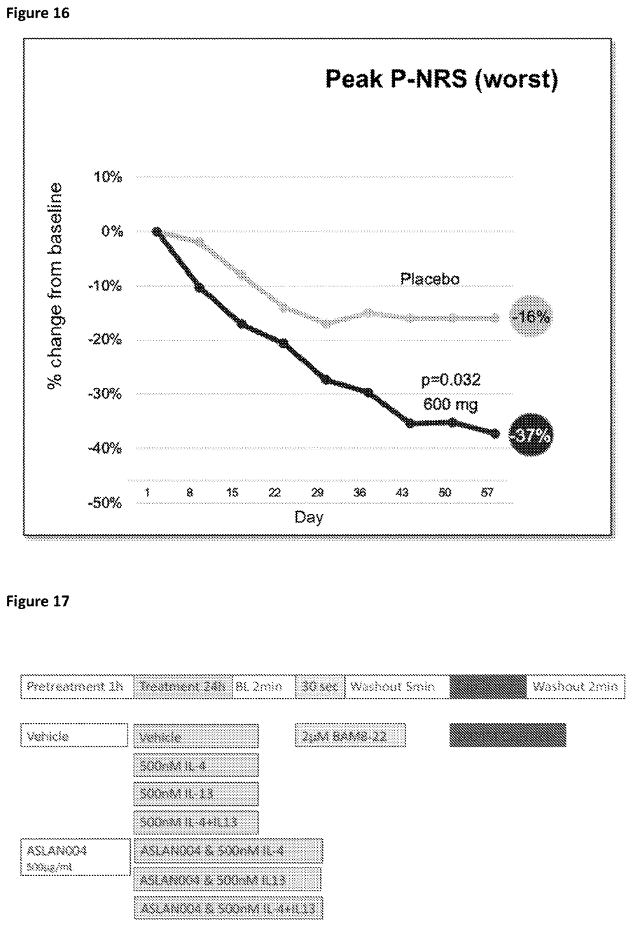

FIG. 13A shows the % change from baseline at day 15, 29, 43 and 57 or all patients receiving treatment. FIG. 13B shows the average % change from baseline over the same period.
FIG. 13C to E shows the data for the individual patients.

TABLE 2
Shows a reduction and −negative number

| | Placebo | Drug |
|---|---|---|
| Study | % cfbl | % cfbl |
| ASLAN004 | 41.6 | −34 |
| 400 mg QW | | |
| Dupilumab | 15 | −35 |
| 300 mg QW Ph2a | | |
| Dupilumab | 10 | −15 |
| 300 mg QW Ph2b/Ph3 | 15 | −30 |

It can be seen that in highly allergic population of patients (IgE 41.6% cfbl) ASLAN004 reduced the IgE levels by −34%. In real terms this is a more significant reduction than the −15 to −30% in a population with lower allergic response in dupilumab.

### Example 2

In the 32 patients that completed at least 29 days of dosing across all sites, defined in the protocol as the efficacy evaluable data set, the average reduction from baseline in EASI at 8 weeks was 73% (n=19) compared to 44% (n=13) for patients on placebo (p=0.0071).
The proportion of patients with adverse events and treatment-related adverse events were similar across treatment and placebo arms. There were no incidences of conjunctivitis in the expansion cohort.

| | RITT (n = 29) | | | ITT (n = 38) | | |
|---|---|---|---|---|---|---|
| | 600 mg | Placebo | p- | 600 mg | Placebo | p- |
| Endpoint | (n = 16) | (n = 13) | value¹ | (n = 22) | (n = 16) | value¹ |
| Mean % | −64.9 | −27.2 | 0.021 | −61.3 | −31.9 | 0.023 |
| change from | | | | | | |
| baseline | | | | | | |
| in EASI | | | | | | |
| EASI-50 (%) | 81.3 | 30.8 | 0.008 | 77.3 | 37.5 | 0.016 |
| EASI-75 (%) | 68.8 | 15.4 | 0.005 | 50.0 | 12.5 | 0.018 |
| EASI-90 (%) | 37.5 | 15.4 | 0.183 | 27.3 | 12.5 | 0.245 |
| IGA 0/1 (%) | 43.8 | 15.4 | 0.107 | 31.8 | 18.8 | 0.301 |
| Mean % | −38.6 | −15.3 | 0.051 | −37.1 | −15.7 | 0.032 |
| change from | | | | | | |
| baseline | | | | | | |
| in peak | | | | | | |
| pruritis | | | | | | |
| Numerical | | | | | | |
| Rating Scale | | | | | | |
| Mean change | −9.8 | −2.5 | 0.007 | −9.0 | −3.5 | 0.014 |
| from baseline | | | | | | |
| in POEM | | | | | | |

¹One-sided p-value

ASLAN004 achieved a statistically significant improvement (p<0.025) versus placebo in the primary efficacy endpoint of percent change from baseline in the Eczema Area Severity Index (EASI), and also showed significant improvements (p<0.05) in other key efficacy endpoints: EASI-50, EASI-75, peak pruritis and the Patient-Oriented Eczema Measure (POEM).

Following discussions with the Data Monitoring Committee prior to unblinding, a Revised ITT population (RITT, n=29) was defined to exclude one study site at which all patients enrolled in the study appeared atypical of moderate-to-severe AD patients based on biomarkers, such as TARC, and patient medical history. In the RITT population, which is more comparable to other published studies in moderate-to-severe AD, ASLAN004 also achieved a statistically significant improvement (p<0.025) versus placebo in percent change from baseline in EASI and showed a greater improvement over placebo in the key efficacy endpoints versus the ITT population.

### Example 3—In Vitro Study on Neuronal Itch Mechanisms by Targeting IL-13Ra1 With Eblasakimab

Chronic itch is a cardinal feature of multiple type-2 driven skin disorders exemplified by atopic dermatitis (AD). The signaling of itch in AD has been recently postulated to be amplified by the inflammatory cytokines present within the skin. In inflammatory skin disease, cytokines exacerbate the immune responses, disrupt the skin barrier, and drive the disease pathology. The direct neuronal modulation by type-2 canonical cytokines was first described with Interleukin-31 (IL-31) which directly activates sensory neurons to signal itch in mice.

The objective is to evaluate different inflammatory scenarios and dissect the role of classical cytokines in their neuro-immune regulation of itch and other somatosensory modularities. Moreover, the present inventors aim to understand the relevance of targeting the IL-13 receptor, IL-13Ra1, on human sensory neurons and how this might result in cellular and intracellular changes altering neuronal activity.

To study these phenomena, the present inventors used eblasakimab, a monoclonal human IgG4 antibody, which binds to the human IL-13Ra1 with high affinity. By binding to the receptor, eblasakimab prevents signaling of IL-4 and IL-13 through the type-2 receptor, which is expressed on a multitude of different immune and non-immune cells except for T-cells.

Using an ex-vivo human neuronal model system, human dorsal root ganglia (hDRG) neurons were treated with IL-4 or IL-13 alone, or in combination, and were subsequently subjected to pruritogens (BAM8-22) that represent histaminergic and non-histaminergic itch. BAM8-22 is a proteolytically cleaved product of proenkephalin A and has been detected in various human tissues. BAM8-22 induces itch across species via activation of the mas-related GPCR (Mrgpr) X1. Neuronal responses were captured by live cell calcium imaging.

### Materials and Methods

### Compounds & Concentrations

IL-13—500 nM
IL-4—500 nM
Eblasakimab—500 μg/mL

### Test Conditions for BAM8-22 Assay

1. BAM control
2. IL-13 (24 h), then BAM
3. IL-4 (24 h), then BAM
4. IL-13+IL-4 (24 h), then BAM
5. Eblasakimab (1 h), Eblasakimab+IL-13 (24 h), then BAM
6. Eblasakimab (1 h), Eblasakimab+IL-4 (24 h), then BAM
7. Eblasakimab (1 h), Eblasakimab+IL-13+IL-4 (24 h), then BAM

### Human DRG Neurons Isolation and Culture

DRGs from 2 donors from the first thoracic vertebra (T1) through the first sacral vertebra (S1) were used in the present study. The DRGs were stripped of connective tissue and enzymatically digested at 37° C. for 2 hrs using the methods described by Davidson et al. Dissociated cells were seeded on 96-well plastic bottom plates (Corning) that had been pre-coated with poly-Dlysine.

Cells were maintained in culture at 37° C. with 5% CO2 in DMEM/F12 supplemented with 10% horse serum (Thermo Fisher Scientific), 2 mM glutamine, 25 ng/ml hNGF (CellSignaling Technology), 25 ng/mL GDNF (Peprotech), Gem21 NeuroPlex (GeminiBio) and penicillin/streptomycin (ThermoFisher Scientific). Half of the culture media was replaced with fresh media every 3 days.

### Live Cell Calcium Imaging

Experiments were conducted on isolated human dorsal root ganglion (hDRG) neurons from post-mortem donors. Cells were loaded with 3 μM Fluo-8-AM (AAT Bioquest) containing 0.1% Pluronic F-127 (Sigma) for 30 min. at room temperature. Extracellular solution contained in (mM): 145 NaCl, 3 KCl, 2 CaCl₂, 1 MgCl₂, 10 HEPES, 10 glucose adjusted to pH 7.4 with NaOH. Fluo-8-loaded cells were excited at 480 nm and emission was collected at 520 nm with a pcoEDGE sCMOS camera (PCO) mounted on an inverted microscope (Olympus IX71). Images were acquired at 0.2 Hz for 10 min in constant perfusion of the extracellular solution adding the adding the agonist and or antagonist in the perfusion. At the end of the agonist/antagonist profiling, an additional perfusion of 200 nM Capsaicin (Sigma) was used to identify the nociceptor hDRG neurons as well as a 50 mM Potassium Chloride to identify the responsive neurons. Image acquisition and data analysis were performed using MetaMorph software (Molecular Devices).

FIG. 17 shows the treatment protocol for the assays. A minimum of 50 DRG neurons were used per assay.

### Results

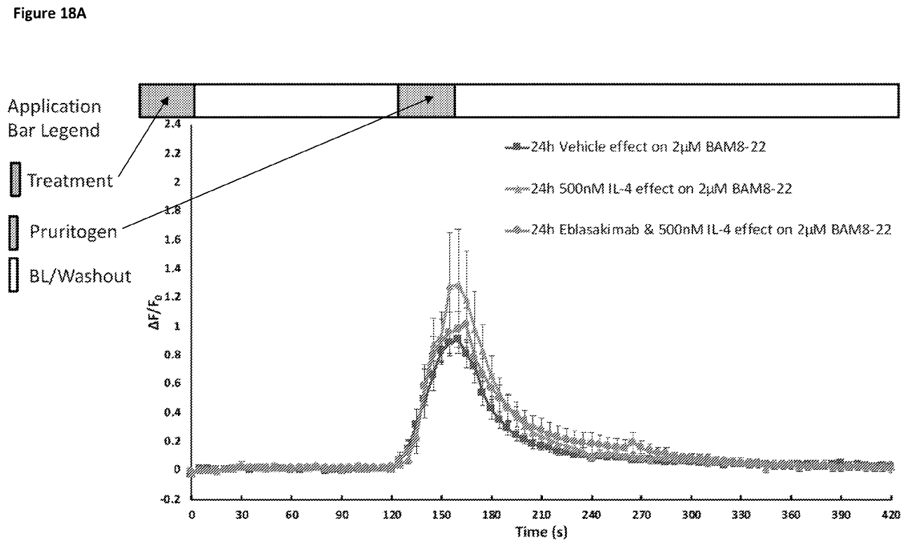

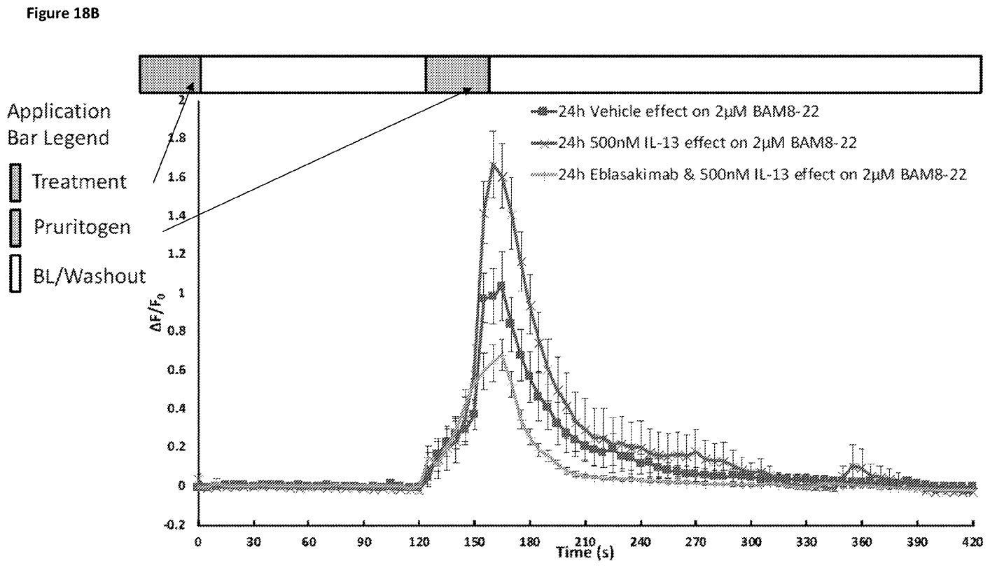

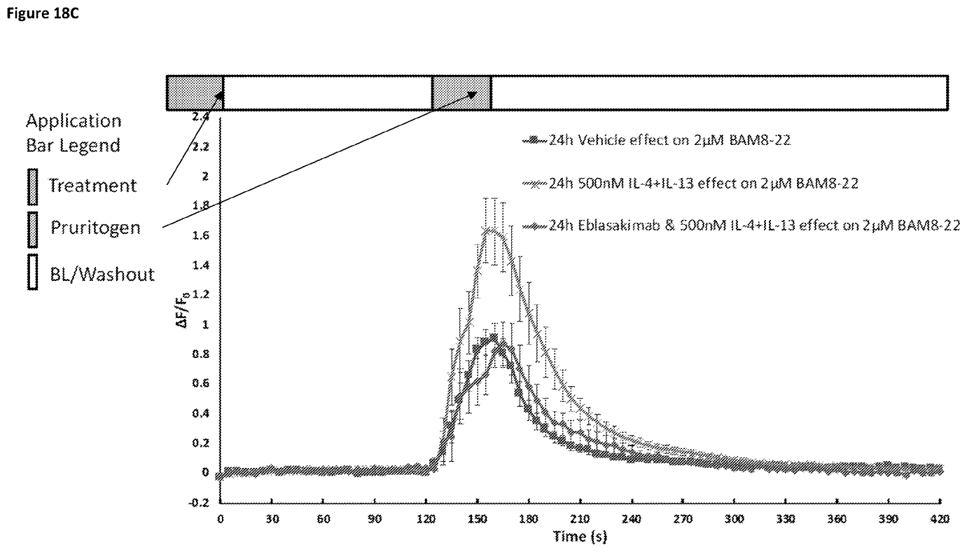

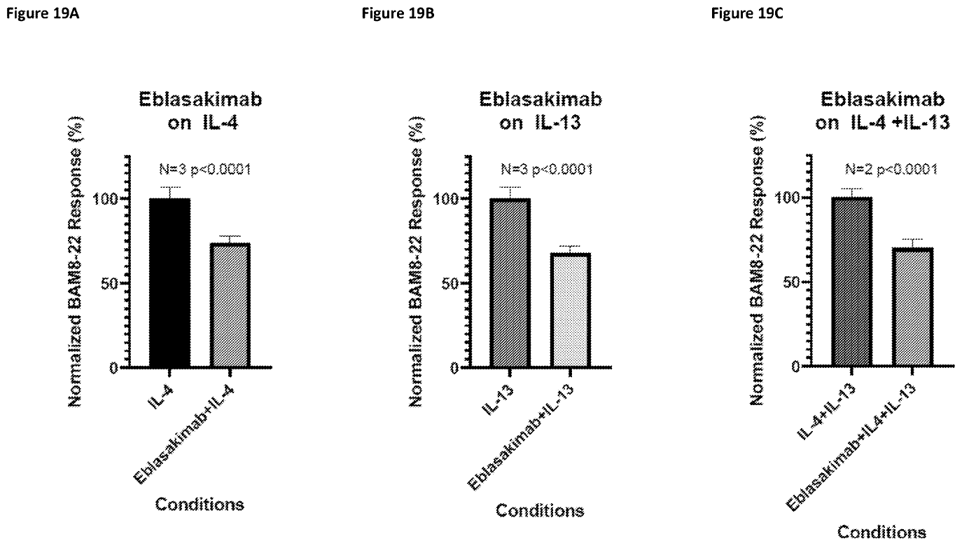

The results are shown in FIGS. 18 to 20.

The data with human sensory neurons pre-stimulated with IL-4, IL-13 and their combination showed a neuronal enhancer effect for IL-4, and IL-13 with no obvious synergy or combined additive effects, which the present inventors postulate may be due to competitive binding and signaling between the two cytokines.

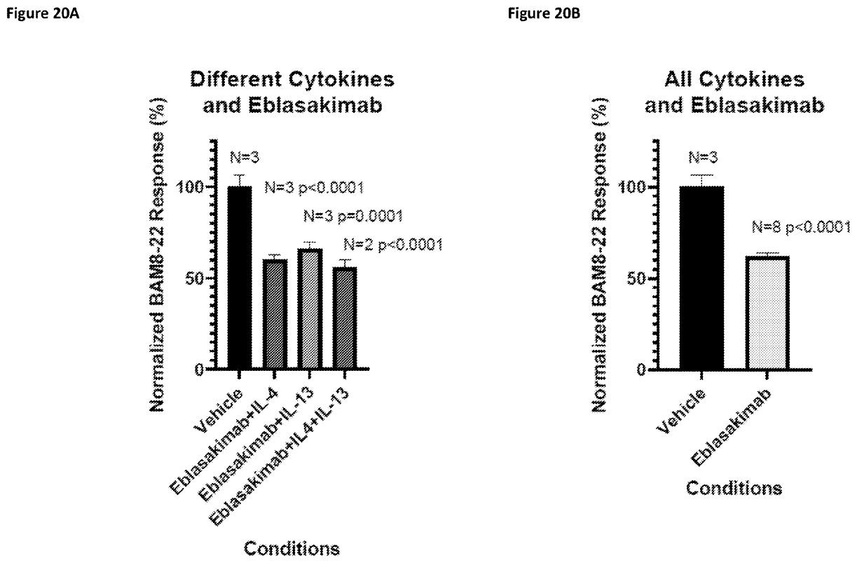

The results further demonstrate that eblasakimab was able to significantly reduce neuronal responses to pruritogens by altering the neuronal sensitization driven by IL-4, IL-13 or a combination of both cytokines. Intriguingly, the itch response in neurons treated with eblasakimab were suppressed to a significantly greater extent than the vehicle control (see FIGS. 20A and B), suggesting a differentiated neuro-regulatory role of IL-13Ra1 signaling beyond the sensitization of neuronal itch pathways.

In contrast to other prior art anti-IL-13R antibodies such as lebrikizumab, eblasakimab can also inhibit IL-4. Thus, unlike lebrikizumab, the present inventors believe that eblasakimab might be able to directly affect neuronal activity to pruritogens via a unique mechanism of action.

In summary, the present inventors have demonstrated that IL-13 and IL-4 act as neuronal enhancers for the amplification of itch pathways through the IL-13Rα1 subunit of the Type-2 receptor and these effects can be inhibited by eblasakimab.

### Example 6—Follow-Up In Vitro Study on Neuronal Itch Mechanisms by Targeting IL-13Ra1 With Eblasakimab

As a follow-up study to Example 5, human dorsal root ganglia (hDRG) neurons were treated with IL-4 or IL-13 and were subsequently subjected to the pruritogen pro-adrenomedullin peptide 1-20 (PAMP20), which is a ligand of the itch specific Mas-related G-protein coupled receptor X2 (MRGPRX2). Neuronal responses were captured by live cell calcium imaging. In addition, neuronal spontaneous activity (SA) induced by IL-4 and IL-13 alone or in combination with inflammatory soup (IS) was measured. Inflammatory soup (IS) is a mix of prostaglandin E2 and bradykinin.

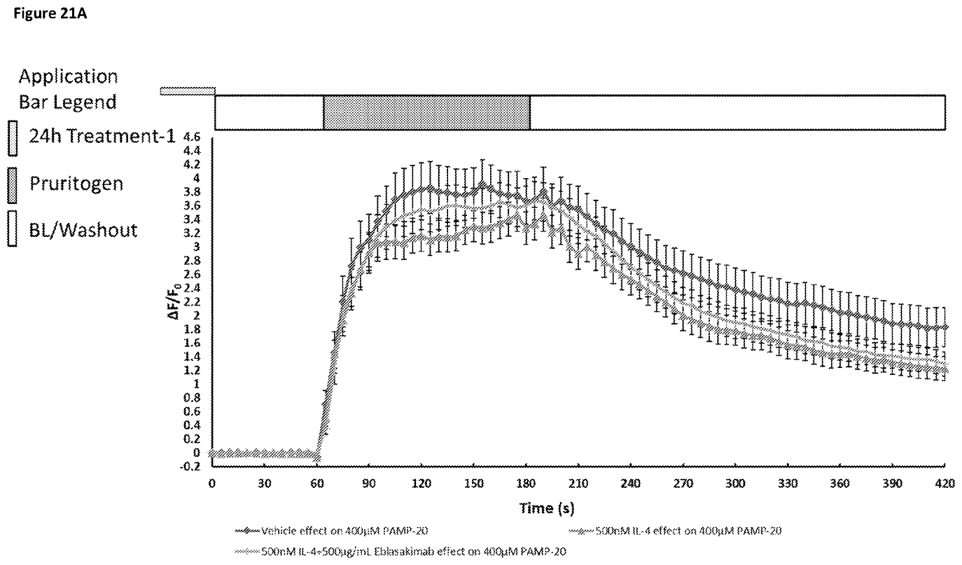

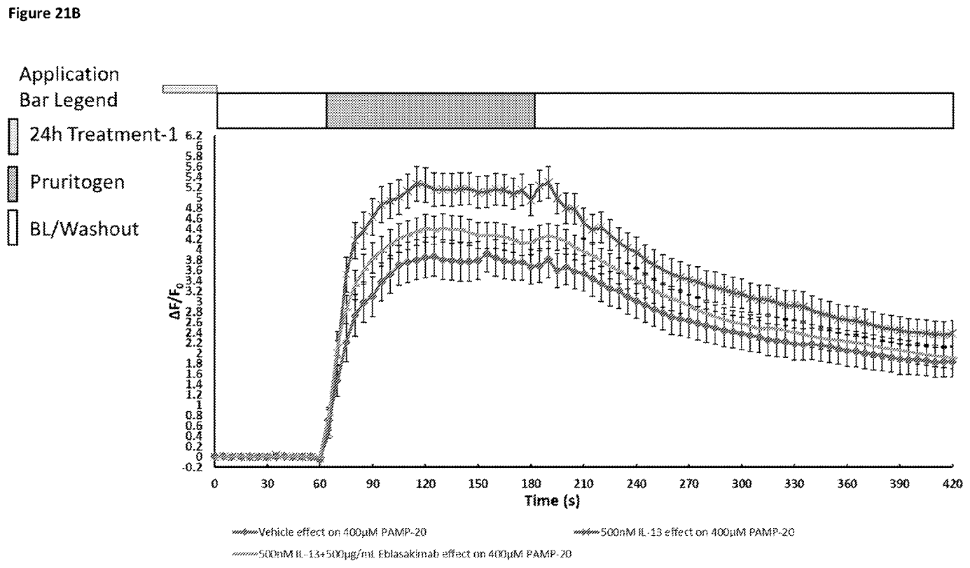

The results of the study are shown in FIGS. 21 to 23.

The results in FIG. 21B indicate that IL-13 sensitized hDRGs to PAMP20, a ligand of MRGPRX2, demonstrating that the itch receptor is expressed on human sensory neurons.

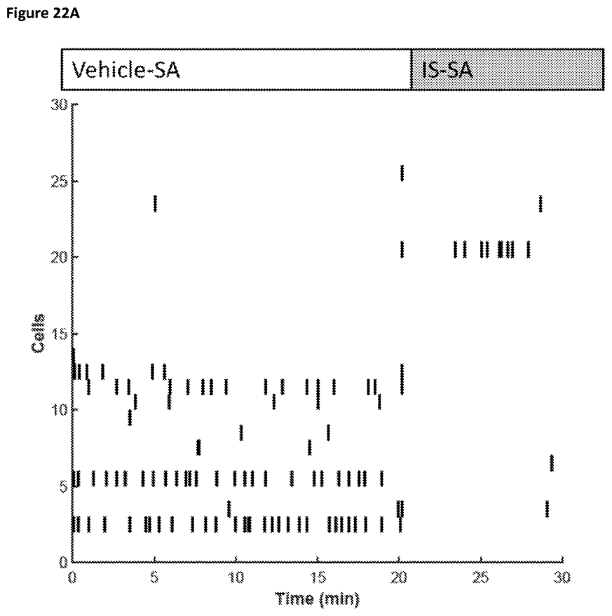

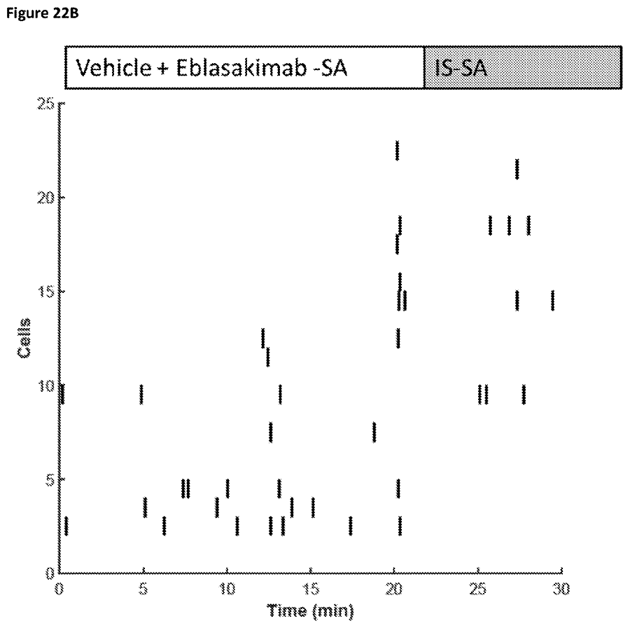

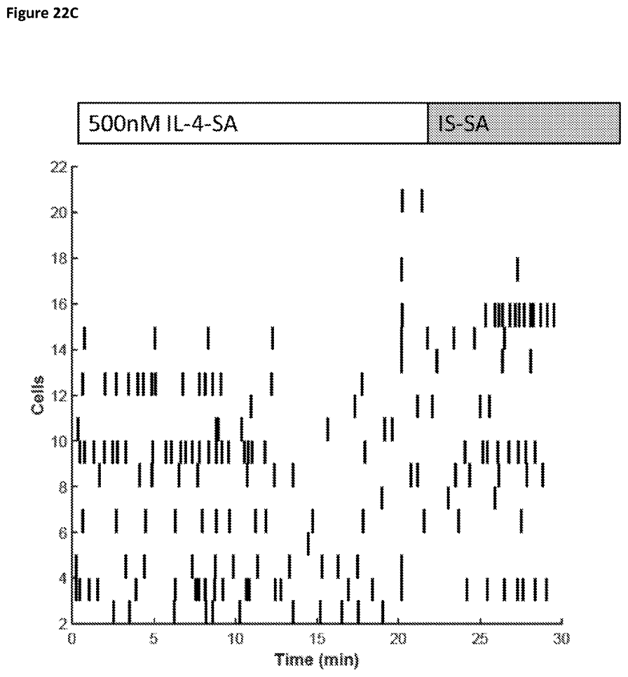

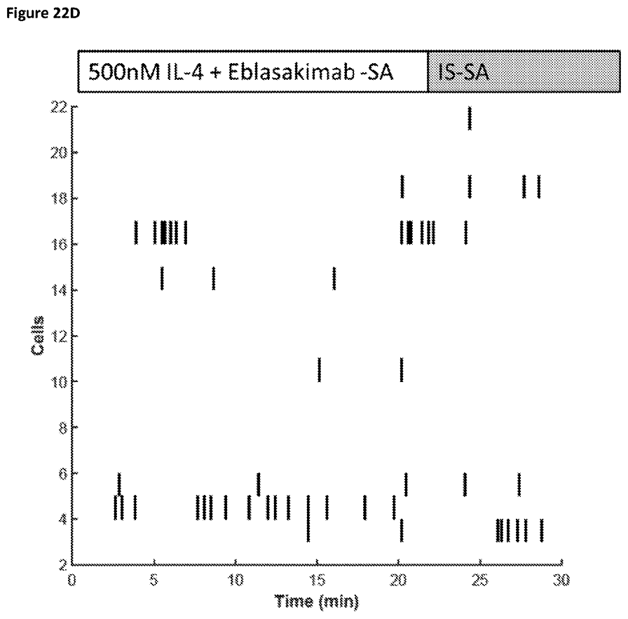

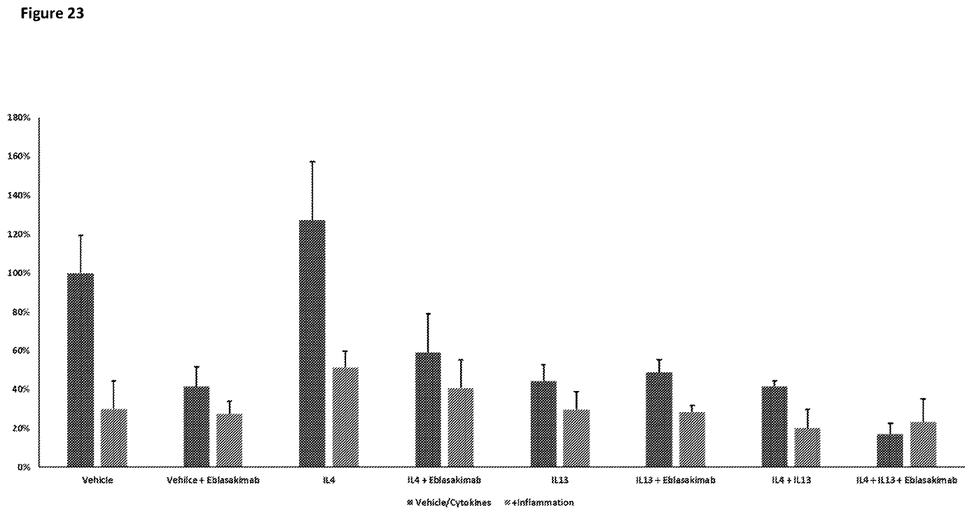

The data on neuronal SA shows that IL-4 resulted in increased cellular activity as compared to vehicle and enhanced inflammatory activity induced by IS. Both enhancement effects were significantly reduced by eblasakimab. See for example FIG. 22C (IL-4 alone) vs FIG. 22D (IL-4+eblasakimab) and FIG. 23.

Thus, this study demonstrates that signaling through IL-13Rα1 may result in neuronal amplification for a wide range of itch pathways, and may expand on neuromodulation beyond itch, which may be targeted by eblasakimab.

### Example 7

The objective of this study was to evaluate the effects of eblasakimab on itch and sleep scores in AD.

### Methods

Three patient cohorts were randomized to receive either 200, 400 or 600 mg eblasakimab or placebo subcutaneously once weekly for 8 weeks in a multiple ascending dose study design.

Adult patients were included with chronic AD present for ≥3 years before screening, and the following AD parameters at screening and baseline: eczema area and severity index (EASI) ≥16, Investigator's Global Assessment (IGA) score ≥3 (scale of 0 to 4), and ≥10% body surface area (BSA) of AD involvement. Rescue medication (moisturizer with active ingredient, topical corticosteroids, topical calcineurin inhibitors) was not allowed; LOCF was used for participants who used rescue medication.

Patient reported outcomes were measured, including pruritus numeric rating scale (P-NRS) for both worst and average itch and Patient-Oriented Eczema Measure (POEM), which includes a single item sleep loss component.

Inferential statistical analysis was performed for 600 mg vs. placebo groups at week 8 only; results for 200 and 400 mg groups were descriptively described due to small sample size.

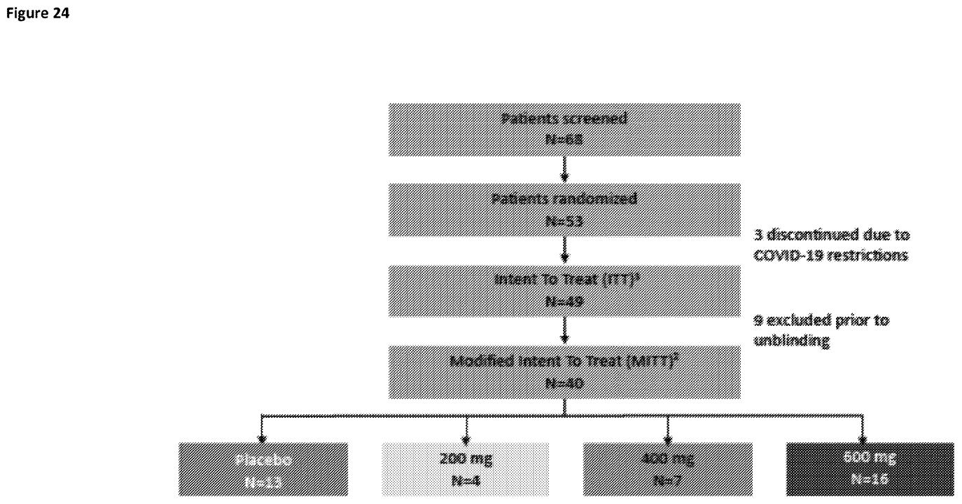

Efficacy analysis in the Phase 1b study used a modified Intent to Treat (mITT) population in which 9 study patients from one site were excluded (excluded site group) from the ITT analysis prior to unblinding as the participants did not have disease characteristics consistent with moderate to severe AD (FIG. 24).

### Results

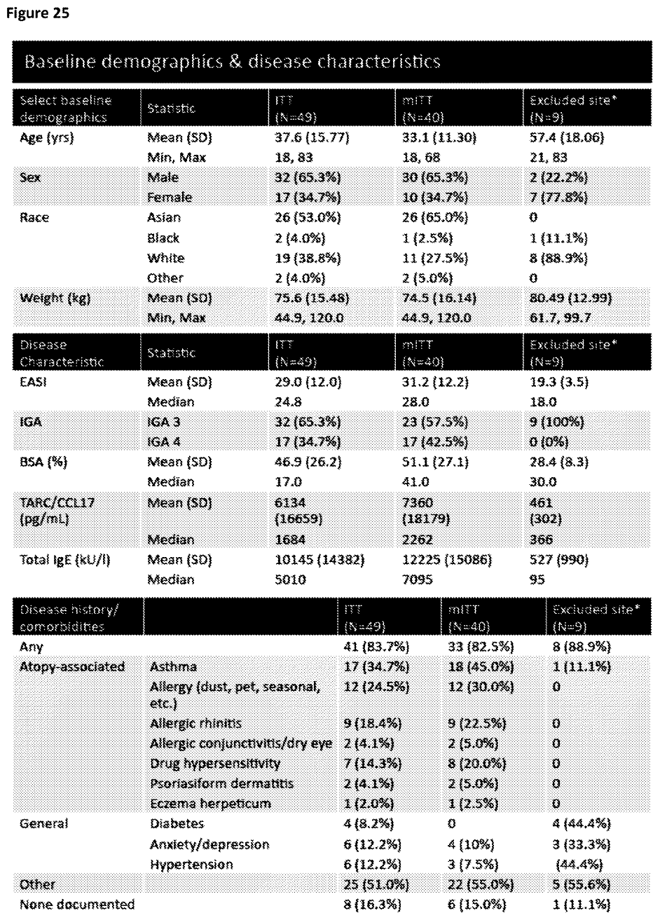

The Excluded site set was markedly different from the mITT set at baseline with substantially lower serum TARC/CCL17 (7,350 pg/mL and 461 pg/mL, respectively), serum IgE (12,225 kU/I vs 527 kU/I), and EASI scores (mean 31.2 vs 19.3) showing lower extent and severity of disease. Other notable differences included older age, and lower IGA, BSA and POEM scores. Participants in this site had no atopic disease history but reported other comorbidities including diabetes and hypertension (FIG. 25).

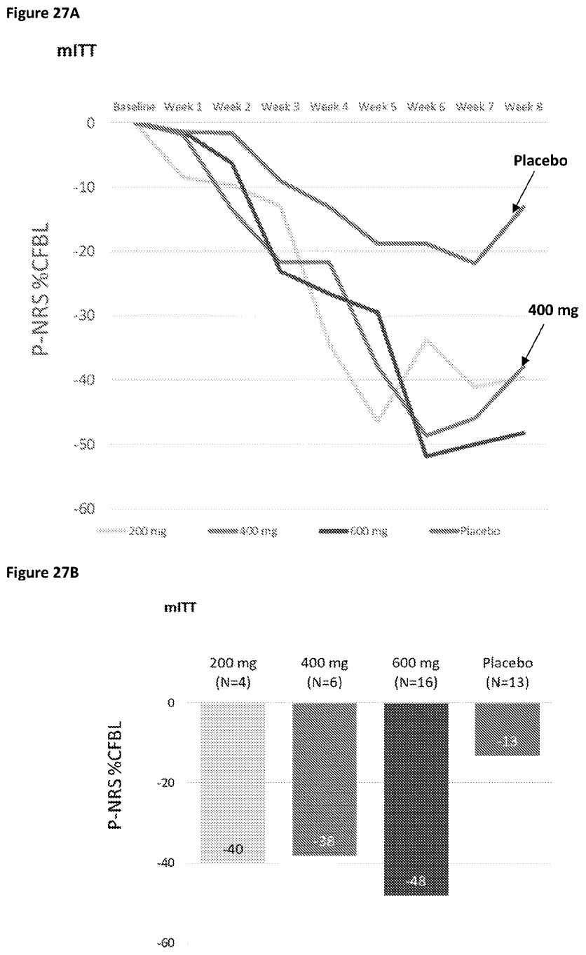

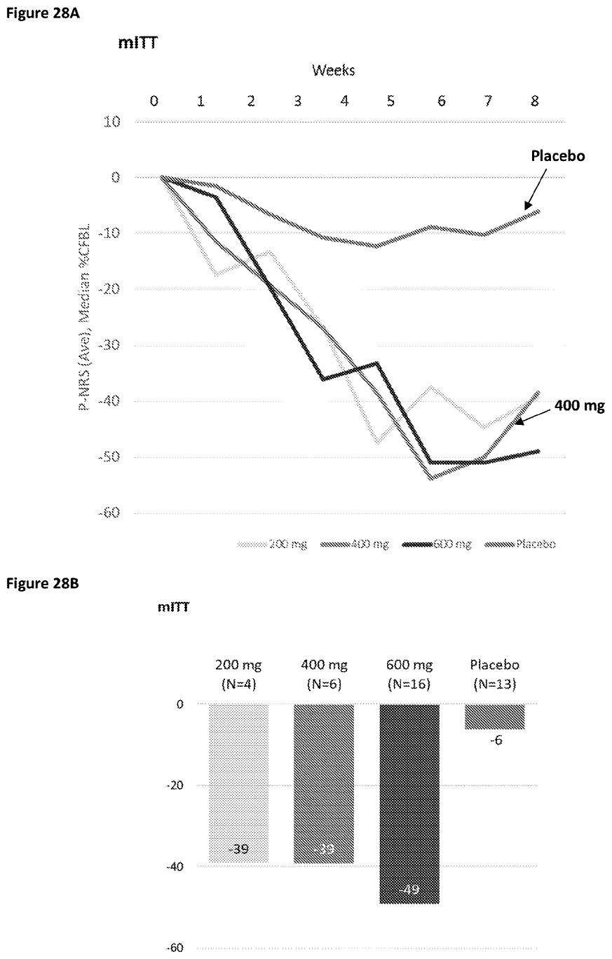

Improvements in percent change from baseline (% CFBL) in P-NRS for median worst itch were apparent over time and at week 8 (FIGS. 27A & 27B) and also for median average itch (FIGS. 28A & 28B) with eblasakimab treatment compared with placebo (worst itch: −48% vs. −13%; average itch: −49% vs. −6%, eblasakimab 600 mg vs. placebo, respectively) in the mITT analysis set.

Participants receiving eblasakimab 600 mg in the Excluded site* exhibited improvement in average itch but not worst itch vs. placebo.

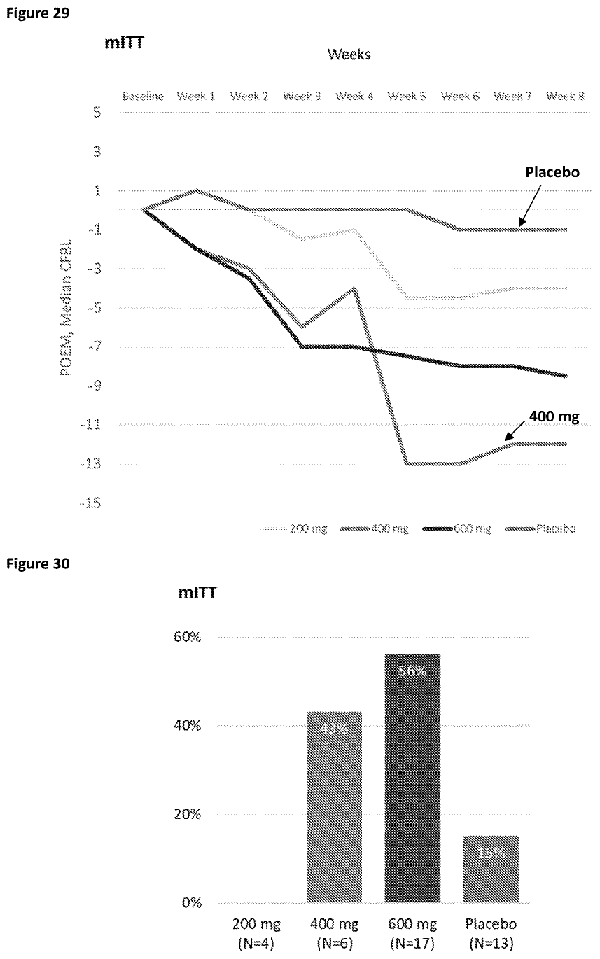

Improvements in POEM score were apparent over time with eblasakimab treatment compared with placebo in the mITT set, with the 400 and 600 mg doses producing a greater magnitude of response than the 200 mg dose (FIG. 29). At week 8, median POEM CFBL for 400 mg and 600 mg eblasakimab was −12 and −9 respectively, vs. −1 for placebo in the mITT set. No improvement vs. placebo was observed in the Excluded site group.

A 4-point improvement in POEM score was observed at week 8 for eblasakimab 600 mg vs. placebo in the mITT but not the Excluded site* analysis sets (81% vs. 23%; 50% vs. 100%, respectively). There was a greater improvement in POEM sleep scores with eblasakimab vs. placebo (FIG. 30). A 2-point improvement (mean) in sleep loss (POEM item) was observed at week 8 for 400 mg and 600 mg eblasakimab (43% and 56% vs. 15% for placebo) in the mITT analysis set, and 600 mg eblasakimab in the Excluded site (33% vs. 0% for placebo).

Importantly, these are improvements for patients with sleep disturbance scores of 3 or 4 at baseline. The majority of patients (75% [12/16]) in the 600 mg treatment group reported >5 nights of sleep disturbance at baseline vs. placebo (54% [7/13]). More (63% [10/16]) of eblasakimab-treated patients reported ‘no days’ or ‘1-2 days’ of sleep disturbance at week 8 vs. placebo (38% [5/13])

Rescue medication use was low, but higher in the placebo group (data not shown).

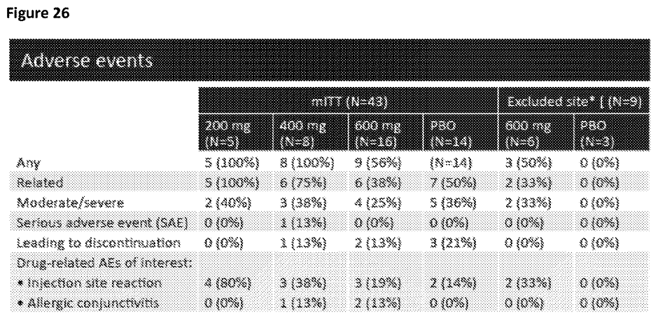

Rates of moderate-to-severe Adverse events (AEs) were comparable between 600 mg and placebo. AEs related to treatment were similar between groups (FIG. 26).

Interestingly, AEs leading to treatment discontinuation were higher in the placebo group. One serious adverse event (SAE) reported in the study (mild abdominal pain, 400 mg); considered unrelated to treatment. No deaths were reported.

### Conclusion

This study indicates that eblasakimab was well tolerated with significant improvements vs. placebo in several efficacy outcomes in a Phase 1b study in adults with moderate to-severe AD.

In particular, a significant reduction in P-NRS for median worst and average itch was observed in patients treated with eblasakimab vs patients treated with placebo.

Robustness of the data from the small study was supported by sensitivity analyses on the primary analysis set. Including the Excluded site data did not change the primary endpoint or conclusions.

That these significant improvements were seen within the 8-week study period offers the potential for a greater magnitude of effect with prolonged treatment, supporting further investigation in an ongoing Phase 2b clinical trial.

1-30. (canceled)

31. A method of treating pruritus in a patient by subcutaneous administration of a treatment cycle comprising a dose in the range 200 mg to 600 mg of an antibody or antigen binding fragment thereof, which is an inhibitor of signalling through IL-13Rα1 by binding the said receptor, wherein the disease is modified by a percentage reduction in pruritus numerical rating score (P-NRS), in the range −15 to −100% from the baseline; and wherein the anti-IL-13R antibody comprises a VH domain comprising an amino acid sequence shown in SEQ ID NO: 51 or a sequence at least 95% identical thereto, and a VL domain comprising an amino acid sequence shown in SEQ ID NO: 53 or a sequence at least 95% identical thereto.

32. A method according to claim 31, wherein the reduction in P-NRS is for average itching (average P-NRS).

33. A method according to claim 31, wherein the reduction in P-NRS is for worst itching (peak or worst P-NRS).

34. A method according to claim 31, wherein the patient is a highly allergic patient, where the baseline IgE levels have been established and are at a level of at least 10,000 KU/L+/−2,000.

35. A method according to claim 31, wherein the pruritus is mediated by IL-13, IL-4 or a combination of both.

36. A method according to claim 31, wherein the patient has a skin condition, selected from the group comprising dermatitis, atopic dermatitis (AD), contact dermatitis, neurodermatitis, seborrheic dermatitis, eczema, hand-foot-and-mouth disease, hives, urticaria associated with Lupus, psoriasis, an infection, a fungal, a bacterial infection, impetigo, folliculitis, an allergic skin reaction, Ehlers-Danlos syndrome, asthma, angioedema, and hereditary angioedema (HAE).

37. A method according to claim 31, wherein the patient has atopic dermatitis (AD).

38. A method according to claim 31, wherein a reduction in P-NRS is present after about two weeks from administration of the first dose, after about four weeks from administration of the first dose, after about six weeks from administration of the first dose, or wherein a reduction in P-NRS is present after about eight weeks from administration of the first dose.

39. A method according to claim 31, wherein multiple doses are administered in a treatment cycle.

40. A method according to claim 31, wherein multiple treatment cycles are administered.

41. A method according to claim 31, wherein following the treatment cycle or cycles and disease modification, maintenance therapy is administered.

42. A method according to claim 41, wherein for the maintenance therapy the same dose is administered less frequently, or a lower dose is administered at the same frequency or less frequently.

43. A method according to claim 31, wherein a loading dose in the range 400 to 900 mg is employed before administration of the treatment cycle.

44. A method according to claim 43, wherein the loading dose is 600 mg.

45. A method according to claim 31, wherein the dose is 400 mg or 600 mg.

46. A method according to claim 31, wherein the reduction in P-NRS score is at least −20%.

47. A method according to claim 31, wherein the reduction in P-NRS score is in the range −20 to −70%.

48. A method according to claim 31, wherein disease modification, occurs by day 8, wherein day 1 is the first administration of the antibody or binding fragment thereof.

49. A method according to claim 31, wherein the disease modification in the range −30 to −100% is achieved by about day 57 following first administration on day 1.


SEQUENCE LISTING (63 sequences)

SEQ ID NO: 1 - protein, 10 aa, synthetic construct
  REGION 1..10: note = CDRH1
GYSFTSYWIG

SEQ ID NO: 2 - protein, 10 aa, synthetic construct
  REGION 1..10: note = CDRH2
VIYPGDSYTR

SEQ ID NO: 3 - protein, 9 aa, synthetic construct
  REGION 1..9: note = CDRH3
  VARIANT 1: note = MISC_FEATURE - Xaa denotes Phe, Met, Gln, Leu or Val
  VARIANT 6: note = MISC_FEATURE - Xaa denotes Ser or Ala
  VARIANT 7: note = MISC_FEATURE - Xaa denotes Phe, Leu, Ala or Met
  SITE 9: note = MISC_FEATURE - Xaa denotes Tyr, Gln, Lys, Arg, Trp, His, Ala, Thr, Ser, Asn orGly
XPNWGXXDX

SEQ ID NO: 4 - protein, 9 aa, synthetic construct
  REGION 1..9: note = CDR H3 SEQUENCE
FPNWGALDQ

SEQ ID NO: 5 - protein, 9 aa, synthetic construct
  REGION 1..9: note = CDR H3 SEQUENCE
VPNMGSLDT

SEQ ID NO: 6 - protein, 9 aa, synthetic construct
  REGION 1..9: note = CDR H3 SEQUENCE
FPNWGSMDA

SEQ ID NO: 7 - protein, 9 aa, synthetic construct
  REGION 1..9: note = CDR H3 SEQUENCE
FPNWGSLDH

SEQ ID NO: 8 - protein, 9 aa, synthetic construct
  REGION 1..9: note = CDR H3 SEQUENCE
MPNWGSFDY

SEQ ID NO: 9 - protein, 9 aa, synthetic construct
  REGION 1..9: note = CDR H3 SEQUENCE
MPNWGSFDT

SEQ ID NO: 10 - protein, 9 aa, synthetic construct
  REGION 1..9: note = CDR H3 SEQUENCE
MPNWGSLDH

SEQ ID NO: 11 - protein, 9 aa, synthetic construct
  REGION 1..9: note = CDR H3 SEQUENCE
MPNWGSFDS

SEQ ID NO: 12 - protein, 9 aa, synthetic construct
  REGION 1..9: note = CDR H3 SEQUENCE
MPNWGSLDT

SEQ ID NO: 13 - protein, 9 aa, synthetic construct
  REGION 1..9: note = CDR H3 SEQUENCE
MPNWGSLDA

SEQ ID NO: 14 - protein, 9 aa, synthetic construct
  REGION 1..9: note = CDR H3 SEQUENCE
MPNWGSLDN

SEQ ID NO: 15 - protein, 9 aa, synthetic construct
  REGION 1..9: note = CDR H3 SEQUENCE
MPNWGALDS

SEQ ID NO: 16 - protein, 9 aa, synthetic construct
  REGION 1..9: note = CDR H3 SEQUENCE
MPNWGSLDN

SEQ ID NO: 17 - protein, 9 aa, synthetic construct
  REGION 1..9: note = CDR H3 SEQUENCE
MPNWGSLDY

SEQ ID NO: 18 - protein, 9 aa, synthetic construct
  REGION 1..9: note = CDR H3 SEQUENCE
MPNWGSFDH

SEQ ID NO: 19 - protein, 9 aa, synthetic construct
  REGION 1..9: note = CDR H3 SEQUENCE
MPNWGSLDS

SEQ ID NO: 20 - protein, 9 aa, synthetic construct
  REGION 1..9: note = CDR H3 SEQUENCE
MPNWGSLDG

SEQ ID NO: 21 - protein, 9 aa, synthetic construct
  REGION 1..9: note = CDR H3 SEQUENCE
VPNWGSLDG

SEQ ID NO: 22 - protein, 28 aa, synthetic construct
  REGION 1..28: note = CDR H3 SEQUENCE
CARFPNWGSLDHWGQGTLVTVSSASIKG

SEQ ID NO: 23 - protein, 28 aa, synthetic construct
  REGION 1..28: note = CDR H3 SEQUENCE
CARMPNWGSLDHWGQGTLVTVSSASTKG

SEQ ID NO: 24 - protein, 28 aa, synthetic construct
  REGION 1..28: note = CDR H3 SEQUENCE
CARMPNWGSFDYWGQGTLVTVSSASIKG

SEQ ID NO: 25 - protein, 12 aa, synthetic construct
  REGION 1..12: note = CDR H3 SEQUENCE
VRMPNWGSLDHW

SEQ ID NO: 26 - protein, 27 aa, synthetic construct
  REGION 1..27: note = CDR H3 SEQUENCE
VRMPNWGSLDHWGQGTLVTVSSADIKG

SEQ ID NO: 27 - protein, 27 aa, synthetic construct
  REGION 1..27: note = CDR H3 SEQUENCE
ARMPNWGSLDHWGQGTLVTVSSASIKG

SEQ ID NO: 28 - protein, 25 aa, synthetic construct
  REGION 1..25: note = CDR H3 SEQUENCE
FPNWGSFDYWGQGTLVTVSSASIKG

SEQ ID NO: 29 - protein, 9 aa, synthetic construct
  REGION 1..9: note = CDR H3 SEQUENCE
VPNWGSLDA

SEQ ID NO: 30 - protein, 9 aa, synthetic construct
  REGION 1..9: note = CDR H3 SEQUENCE
FPNWGSFDY

SEQ ID NO: 31 - protein, 12 aa, synthetic construct
  REGION 1..12: note = CDRL1
RASQSISSSYLA

SEQ ID NO: 32 - protein, 7 aa, synthetic construct
  REGION 1..7: note = CDRL2
GASSRAT

SEQ ID NO: 33 - skipped

SEQ ID NO: 34 - protein, 5 aa, synthetic construct
  REGION 1..5: note = CDRL3 SEQUENCE
QRYAT

SEQ ID NO: 35 - protein, 5 aa, synthetic construct
  REGION 1..5: note = CDRL3 SEQUENCE
QRYST

SEQ ID NO: 36 - protein, 5 aa, synthetic construct
  REGION 1..5: note = CDRL3 SEQUENCE
QMYST

SEQ ID NO: 37 - protein, 5 aa, synthetic construct
  REGION 1..5: note = CDRL3 SEQUENCE
QQVGT

SEQ ID NO: 38 - protein, 5 aa, synthetic construct
  REGION 1..5: note = CDRL3 SEQUENCE
QQVST

SEQ ID NO: 39 - protein, 5 aa, synthetic construct
  REGION 1..5: note = CDRL3 SEQUENCE
QQYST

SEQ ID NO: 40 - protein, 5 aa, synthetic construct
  REGION 1..5: note = CDRL3 SEQUENCE
QSYST

SEQ ID NO: 41 - protein, 5 aa, synthetic construct
  REGION 1..5: note = CDRL3 SEQUENCE
QQYAT

SEQ ID NO: 42 - protein, 5 aa, synthetic construct
  REGION 1..5: note = CDRL3 SEQUENCE
QQYSS

SEQ ID NO: 43 - protein, 5 aa, synthetic construct
  REGION 1..5: note = CDRL3 SEQUENCE
QTYST

SEQ ID NO: 44 - protein, 5 aa, synthetic construct
  REGION 1..5: note = CDRL3 SEQUENCE
QQYGS

SEQ ID NO: 45 - protein, 5 aa, synthetic construct
  REGION 1..5: note = CDRL3 SEQUENCE
QQYAS

SEQ ID NO: 46 - protein, 5 aa, synthetic construct
  REGION 1..5: note = CDRL3 SEQUENCE
QQYEA

SEQ ID NO: 47 - protein, 5 aa, synthetic construct
  REGION 1..5: note = CDRL3 SEQUENCE
QQYET

SEQ ID NO: 48 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARFPNWGSFDYWGQGTLVTVSS

SEQ ID NO: 49 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSS

SEQ ID NO: 50 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSS

SEQ ID NO: 51 - protein, 118 aa, synthetic construct
  REGION 1..118: note = VH region
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSS

SEQ ID NO: 52 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEI

SEQ ID NO: 53 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEI

SEQ ID NO: 54 - protein, 103 aa, synthetic construct
  REGION 1..103: note = VL region
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEI

SEQ ID NO: 55 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 56 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 57 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCVRMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 444 aa, synthetic construct
  REGION 1..444: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASIKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - protein, 443 aa, synthetic construct
  REGION 1..443: note = heavy chain
EVQLVQSGAEVKKPGESLKISCKGSGYSFTSYWIGWVRQMPGKGLEWMGVIYPGDSYTRYSPSFQGQVTISADKSISTAYLQWSSLKASDTAMYYCARMPNWGSLDHWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 61 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYASFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYEAFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 211 aa, synthetic construct
  REGION 1..211: note = light chain
EIVLTQSPGTLSLSPGERATLSCRASQSISSSYLAWYQQKPGQAPRLLIYGASSRATGIPDRFSGSGSGTDFTLTISRLEPEDFAVYYCQQYETFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC